(19) United States
(12) Patent Application Publication
(10) Pub. No.: US 2023/0141532 A1
(43) Pub. Date: May 11, 2023

(54) CHEESE AND YOGURT LIKE COMPOSITIONS AND RELATED METHODS

(71) Applicant: New Culture Inc., San Leandro, CA (US)

(72) Inventors: Matt Gibson, Oakland, CA (US); Inja Radman, Oakland, CA (US)

(21) Appl. No.: 18/055,016

(22) Filed: Nov. 14, 2022

Related U.S. Application Data
(63) Continuation of application No. 17/516,273, filed on Nov. 1, 2021.
Application No. 17/516,273 is a continuation of application No. PCT/US2020/031177, filed on May 1, 2020.
(60) Provisional application No. 62/842,469, filed on May 2, 2019.

(51) Int. Cl. A23C 19/09; A23L 33/19; A23J 3/20; A23C 11/10; A23C 20/02

(52) U.S. Cl. CPC A23C 19/0917; A23L 33/19; A23J 3/20; A23C 11/10; A23C 20/02; A23C 2220/202; A23C 2250/10

(57) ABSTRACT

Provided herein are cheese and yogurt compositions and the methods of making the same using one or more recombinant proteins.

### CROSS-REFERENCE

This application is a continuation application of U.S. application Ser. No. 17/516,273, filed Nov. 1, 2021, which is a continuation application of International Application No. PCT/US2020/031177, filed on May 1, 2020, which application claims the benefit of U.S. Provisional Application No. 62/842,469, filed on May 2, 2019, which applications are incorporated herein by reference in their entirety.

### BACKGROUND OF THE INVENTION

The clean food space is comprised of both plant-based and cell-based foods. Cell-based food is a large umbrella term that includes culturing muscle and fat cells to replace slaughtered meat and culturing bioengineered organisms to express recombinant animal proteins to replace other animal products such as dairy and eggs. The need to find an alternate source of animal protein comes from the inefficiencies and unsustainability of current animal food production.

Cheese is the third most unsustainable animal product globally (when measuring greenhouse gas emissions per kg of product), and the consumption of dairy cheese hasn't been slowed down by plant-based alternatives introduced into the market in the last 10 years. On the contrary, mozzarella cheese consumption is growing year on year in the US and in developing markets. Current cheese alternatives do not match the functionality, nutrition and taste of dairy cheese due to their lack of casein proteins.

One common trait that all companies in this space so far have shared is the difficulty to scale at pace and at affordable cost. Recombinant protein production can be very expensive and slow. This is partially because the downstream costs of protein purification can reach up to 80% of the entire protein production processing costs and the reduction in protein yield can be as high as 70% depending on the purity of the product.

### SUMMARY OF THE INVENTION

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

In some aspects, described herein are cheese compositions. A cheese composition may comprise a coagulated colloid, wherein the coagulated colloid comprises alpha casein protein and kappa casein protein associated in a micellar form. At least one of the alpha casein protein and the kappa casein protein may be recombinantly produced; and wherein the cheese composition may not contain beta casein protein.

In some embodiments, the recombinantly produced casein may be produced from a bacterial host cell.

In some embodiments, the alpha and kappa casein proteins are both recombinantly produced.

In some embodiments, the recombinantly produced alpha and kappa casein proteins are produced from one or more bacterial host cells.

In some embodiments, the alpha casein protein completely lacks or may be substantially reduced in post-translational modification as compared to native alpha casein.

In some embodiments, the alpha casein protein completely lacks or may be substantially reduced in phosphorylation as compared to native alpha casein.

In some embodiments, the kappa casein protein completely lacks or may be substantially reduced in post-translational modification as compared to native kappa casein.

In some embodiments, the kappa casein protein completely lacks or may be substantially reduced in glycosylation as compared to native kappa casein.

In some embodiments, the kappa casein protein completely lacks or may be substantially reduced in phosphorylation as compared to native kappa casein.

In some embodiments, the bacterial host cell may be selected from the group consisting of Lactococci sp., Lactococcus lactis, Bacillus subtilis, Bacillus amyloliquefaciens, Bacillus licheniformis and Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum, Synechocystis sp. 6803 and E. coli.

In some embodiments, the bacterial host cell secretes the recombinantly produced casein protein.

In some embodiments, the bacterial host retains the recombinantly produced casein protein intracellularly.

In some embodiments, the production of the recombinantly produced protein in the bacterial host cell may be regulated by an inducible promoter.

In some embodiments, the production of the recombinantly produced protein in the bacterial host cell may be regulated by a constitutive promoter.

In some embodiments, the ratio of alpha casein protein to kappa casein protein may be between about 1:1 and about 15:1. In some embodiments, the ratio may be about 1:1, 2:1, 3:1, 4:1, 5:1, 6:1, 7:1, 8:1, 9:1, 10:1, 11:1, 12:1, 13:1, 14:1 or 15:1.

In some embodiments, the alpha casein protein may be alpha s1 or alpha s2.

In some embodiments, the alpha casein protein may be encoded by a protein sequence selected from SEQ ID NO. 1-26 or a variant with at least 80% sequence homology.

In some embodiments, the kappa casein protein may be encoded by a protein sequence selected from SEQ ID NO. 27-40 or a variant with at least 80% sequence homology.

In some embodiments, the cheese composition comprises a population of the micellar forms sized between about 150 nm to about 500 nm or between about 100 nm to about 500 nm.

In some embodiments, a portion of the micellar forms of the population may be sized less than 100 nm or between about 10 nm and 100 nm.

In some embodiments, the cheese may comprise at least one salt, selected from the group consisting of a calcium salt, a citrate salt and a phosphate salt. In some embodiments, the cheese lacks any additional dairy-derived proteins.

In some embodiments, the cheese lacks any animal-derived dairy proteins.

In some embodiments, the cheese has a fat content between about 0% to about 50% and the fat may be derived from a plant-based source.

In some embodiments, the cheese has a sugar content between about 0% to about 10% and the sugar may be derived from a plant-based source.

In some embodiments, the cheese may be capable of melting and browning when heated.

In some embodiments, the cheese may be selected from the group consisting of pasta-filata like cheese, paneer, cream cheese and cottage cheese.

In some embodiments, the cheese may be mozzarella.

In some embodiments, the cheese may be an aged or matured cheese selected from the group consisting of cheddar, swiss, brie, camembert, feta, halloumi, gouda, edam, cheddar, manchego, swiss, colby, muenster, blue cheese or parmesan.

In some embodiments, the moisture retention of the cheese may be 40-65%.

In some embodiments, the texture of the cheese may be comparable to an animal-derived dairy cheese.

In some embodiments, the hardness of the cheese may be comparable to an animal-derived dairy cheese.

In some aspects, described herein are methods of producing an edible composition. The methods for producing an edible composition, may comprise: combining a recombinant alpha casein protein, a recombinant kappa casein protein and at least one salt under conditions wherein the alpha casein protein and the kappa casein protein form a micellar form in a liquid colloid, wherein the micellar form does not include beta casein protein; and subjecting the liquid colloid to a first condition to form coagulates.

In some embodiments, the first condition may be the addition of acid or acidification of the liquid colloid with a microorganism.

In some embodiments, the method further comprises subjecting the coagulates to a hot water treatment and optionally stretching, to form a filata-type cheese.

In some embodiments, the method further comprises subjecting the coagulates to a renneting agent to form a rennetted curd.

In some embodiments, the renneting agent may be a microbially-derived chymosin enzyme.

In some embodiments, the method further comprises aging and maturing the rennetted curd to form a cheese-like composition.

In some embodiments, the method further comprises subjecting the rennetted curd to a hot water treatment and optionally stretching, to form a filata-type cheese.

In some embodiments, the edible composition does not include beta casein protein.

In some embodiments, the edible composition does not include any additional dairy-derived protein.

In some embodiments, the edible composition does not include any animal-derived dairy protein.

In some embodiments, the recombinantly produced alpha and kappa casein proteins are produced from one or more bacterial host cells.

In some embodiments, the alpha casein protein completely lacks or may be substantially reduced in phosphorylation as compared to native alpha casein.

In some embodiments, the kappa casein protein completely lacks or may be substantially reduced in glycosylation as compared to native kappa casein.

In some embodiments, the kappa casein protein completely lacks or may be substantially reduced in phosphorylation as compared to native kappa casein.

In some embodiments, the method does not comprise treatment of the alpha casein protein and/or the kappa casein with enzymes that modulate post-translational modification.

In some embodiments, the bacterial host cell may be selected from the group consisting of Lactococci sp., Lactococcus lactis, Bacillus subtilis, Bacillus amyloliquefaciens, Bacillus licheniformis and Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum, Synechocystis sp. 6803 and E. coli.

In some embodiments, one or more bacterial host cells secrete the recombinantly produced alpha casein protein and kappa casein protein.

In some embodiments, one or more bacterial host cells retain the recombinantly produced alpha casein protein and kappa casein protein.

In some embodiments, the production one or both alpha casein protein and kappa casein protein may be regulated by an inducible promoter.

In some embodiments, the production one or both alpha casein protein and kappa casein protein may be regulated by a constitutive promoter.

In some embodiments, the ratio of alpha casein protein to kappa casein protein in the micellar form may be between about 1:1 and about 15:1.

In some embodiments, the ratio may be about 1:1, 2:1, 3:1, 4:1, 5:1, 6:1, 7:1, 8:1, 9:1, 10:1, 11:1, 12:1, 13:1, 14:1 or 15:1.

In some embodiments, the alpha casein protein may be alpha s1 or alpha s2.

In some embodiments, the alpha casein protein may be encoded by a nucleotide sequence selected from SEQ ID NO. 1-26 or a variant with at least 80% sequence homology.

In some embodiments, the kappa casein protein may be encoded by a nucleotide sequence selected from SEQ ID NO. 27-40 or a variant with at least 80% sequence homology.

In some embodiments, the liquid colloid comprises a population of the micellar forms sized between about 150 nm to about 500 nm or between about 100 nm to about 500 nm

In some embodiments, a portion of the micellar forms of the population may be sized less than 100 nm or between about 10 nm and 100 nm.

In some embodiments, a salt in the liquid colloid may be a calcium salt.

In some embodiments, the step of forming the liquid colloid further comprises the addition of phosphate and/or citrate.

The method of claim 33, wherein the renneting coagulation time may be from 1 minute to 6 hours.

In some aspects, described herein are liquid colloid micellar compositions. The liquid colloid may comprise a micellar form, wherein the micellar form comprises a recombinant alpha casein protein, a recombinant kappa casein protein and at least one salt, and wherein the alpha casein protein, the kappa casein protein or a combination thereof completely lack or are substantially reduced in post-translational modifications.

In some embodiments, (a) the alpha casein protein completely lacks or may be substantially reduced in phosphorylation as compared to native alpha casein, or (b) the kappa casein protein completely lacks or may be substantially reduced in glycosylation as compared to native kappa casein, or (c) the kappa casein protein completely lacks or may be substantially reduced in phosphorylation as compared to native kappa casein, or (d) including (a), (b) and (c) together.

In some embodiments, the micellar form does not include beta casein protein.

In some embodiments, a yogurt composition may be formed using the methods described herein. The yogurt may be formed using the liquid colloid described herein. The method may comprise heating and then cooling the liquid colloid and acidifying the liquid colloid with a microorganism. The microorganism may comprise one or more of Lactobacillus delbrueckii subsp. bulgaricus, Streptococcus thermophilus, a lactobacilli or a bifidobacteria.

In some embodiments, the yogurt composition may be formed by the methods described herein, wherein the α casein protein comprises an amino acid sequence of cow, human, sheep, goat, buffalo, bison, horse or camel α casein protein.

In some embodiments, the yogurt composition may be formed by the methods described herein, wherein the κ casein protein comprises an amino acid sequence of cow, human, sheep, goat, buffalo, bison, horse or camel κ casein protein

In some aspects, provided herein may be a composition comprising: a concentrate of a first growth medium, wherein the concentrate comprises at least one recombinant casein protein; wherein the first growth medium may be compatible for supporting growth of a recombinant microorganism expressing and secreting the at least one recombinant casein protein; and wherein the first growth medium comprises a non-dairy non-animal derived serum; and at least one lactic acid bacteria species, wherein the composition may be compatible for growth of the at least one lactic acid bacteria species.

In some embodiments, provided herein may be a composition comprising: a first growth medium comprising a non-dairy non-animal derived serum, wherein the first growth medium may be compatible for supporting growth of a recombinant microorganism expressing and secreting at least one recombinant casein protein; and wherein a concentrate of the first growth medium may be compatible for growth of at least one lactic acid bacteria species and for forming a cheese-like consistency.

In some embodiments, the composition further comprises at least one recombinant casein protein. In some embodiments, the at least one recombinant casein protein may be selected from the group consisting of alpha casein, beta casein, and kappa casein.

In some embodiments, the composition comprises two recombinant casein proteins.

In some embodiments, the concentrate of the first growth medium comprises micelles and wherein the micelles comprise at least one recombinant casein protein.

In some embodiments, the recombinant microorganism may be a gram-positive bacterium.

In some embodiments, the lactic bacteria species may be a Lactococcus sp.

In some embodiments, the first growth medium may be capable of supporting growth of the recombinant microorganism to near or at stationary phase.

In some aspects, described herein may be a fermented dairy-like product. The fermented dairy-like product may be a product selected from hard cheese, soft cheese, curd cheese, cheese spread, and yogurt.

In some aspects, described herein may be a method for making a fermented dairy-like product comprising: growing a recombinant microorganism expressing a recombinant casein protein in a non-dairy non-animal derived serum, wherein the casein protein may be secreted into the serum; removing the microorganism from the serum; combining the serum with at least one lactic acid bacteria species; whereby after an incubation period, the combination of the serum and the at least one lactic acid bacteria species creates a fermented dairy-like product.

In some embodiments, the serum may be concentrated prior to adding the lactic acid bacteria.

In some embodiments, the recombinant microorganism may be a gram-positive bacterium.

In some embodiments, the recombinant microorganism may be a yeast.

In some embodiments, the recombinant microorganism may be a Lactococcus sp.

In some embodiments, the step of growing comprises growing the recombinant microorganism to near or at stationary phase.

In some embodiments, the fermented dairy-like product may be selected from the group consisting of hard cheese, soft cheese, curd cheese, cheese spread, and yogurt.

### INCORPORATION BY REFERENCE

All publications, patents, and patent applications mentioned in this specification are herein incorporated by reference to the same extent as if each individual publication, patent, or patent application was specifically and individually indicated to be incorporated by reference

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1 is a basic technical diagram of the production process.

FIG. 2 illustrates an exemplary protocol for cheese production.

FIGS. 3A and 3B illustrate expression systems.

FIG. 4A shows yogurt-like gel and curd.

FIG. 4B illustrates curds made from micellar casein liquid colloid (supplemented with lactose) (left) and fat-free milk (right) via microbial acidification using bacterial starter culture. Firm, cuttable and cohesive curd was made in both cases. Bottom left: An example of curd made from micellar liquid colloid (supplemented with lactose) emulsified with deflavoured coconut oil via microbial acidification using bacterial starter culture.

FIG. 4C illustrates cheese and curd firmness (in g force used in stress-relaxation test) and relaxation for mozzarella-like cheese made from microbial casein liquid colloid vs fat-free milk using bacterial starter culture vs citric acid.

FIG. 5A illustrates texture profile and hardness/firmness (in g force) of mozzarella cheese.

FIG. 5B illustrates samples of mozzarella made from micellar casein melted and served on a ‘pizza’ for triangle test tasting.

FIG. 6 illustrates average micelle diameter (in nm) of casein micelles (black) and submicelles (gray) induced using alpha casein, beta-casein and kappa casein in various salt conditions. Relative intensity proportions of micelle (black) and submicelle (gray) peaks detected are represented as arch sizes/angles.

FIG. 7A illustrates liquid milk-like colloids comprised of casein micelles induced using alpha casein, beta-casein and kappa casein in various salt conditions.

FIG. 7B shows liquid milk-like colloids subjected to acidification via citric acid (top row) and renneting coagulation via recombinant chymosin (middle row), which gave rise to dairy curds (bottom row, X where data was not collected). Samples B and F precipitated during acidification and renneting and formed curds on the bottom of the well. All other samples stayed in suspension during acidification and formed curds throughout. Curds were then dipped in hot water and stretched into pasta-filata cheese balls.

FIG. 8 shows average micelle diameter (in nm) of casein micelles (black) and submicelles (gray) induced using alpha casein and kappa casein in various salt conditions. Relative intensity proportions of micelle (black) and submicelle (gray) peaks detected are represented as arch sizes/angles.

FIG. 9A shows hardness (in g force applied) of cheese made from liquid milk-like colloid of casein micelles assembled from alpha casein and kappa casein. Sodium caseinate (a source of mixed milk caseins) was used as a control. Error bars represent standard deviation on triplicate sample.

FIG. 9B shows cheese made from liquid milk-like colloid of casein micelles assembled from alpha casein and kappa casein (in triplicate).

FIG. 10 shows liquid milk-like colloids of casein micelles induced using hypophosphorylated alpha casein (enzymatically dephosphorylated) and kappa casein in various salt conditions were subjected to acidification via citric acid (top row) and renneting coagulation via recombinant chymosin, which gave rise to dairy curds (bottom row, X where the curd was already used for cheese making) Samples F precipitated during acidification and renneting and formed curd on the bottom of the well. All other samples stayed in suspension during acidification and formed curds throughout. Curds were then dipped in hot water and stretched into pasta-filata cheese balls.

FIG. 11 shows cheese made from liquid milk-like colloid of casein micelles assembled from hypophosphorylated alpha casein (enzymatically dephosphorylated) and kappa casein in various salt conditions.

FIG. 12 shows average micelle diameter (in nm) of casein micelles (black) and submicelles (gray) induced using hypophosphorylated (enzymatically dephosphorylated) alpha casein and kappa casein in salt conditions at milk-like protein concentrations (2.8% total casein, 3.2% total casein). Relative intensity proportions of micelle (black) and submicelle (gray) peaks detected are represented as arch sizes/angles.

FIG. 13 shows liquid milk-like colloids comprised of casein micelles induced using alpha casein and deglycosylated kappa casein, or alpha casein and kappa casein. Alpha-casein is kept constant, whereas kappa casein is increased 2-fold and 3-fold.

FIG. 14 shows average micelle diameter (in nm) of casein micelles induced using alpha casein and deglycosylated kappa casein, or alpha casein and kappa casein. Alpha-casein is kept constant, whereas kappa casein is increased 2-fold and 3-fold. Error bars represent standard deviation on triplicate sample.

FIG. 15 shows curds made from acidification and renneting of liquid colloids comprised of casein micelles induced using alpha casein and deglycosylated kappa casein, or alpha casein and kappa casein. Alpha-casein is kept constant, whereas kappa casein is increased 2-fold and 3-fold. All curds formed were strong enough to be inverted without deforming, except for top left sample which formed aggregates.

FIG. 16 shows wet yield (in mg) of cheese made from curds of casein micelles induced using alpha casein and deglycosylated kappa casein, or alpha casein and kappa casein. Alpha-casein is kept constant, whereas kappa casein is increased 2-fold and 3-fold. Cheese (mozzarella) was made by dipping the curd in hot water, stretching and shaping to a cheese ball.

FIG. 17 shows average micelle diameter (in nm) of casein micelles induced using hypophosphorylated alpha casein (enzymatically dephosphorylated) and deglycosylated kappa casein, or hypophosphorylated alpha casein (enzymatically dephosphorylated) and kappa casein. Alpha-casein is kept constant, whereas kappa casein is increased 2-fold. Error bars represent standard deviation on triplicate sample.

FIG. 18 shows average micelle diameter (in nm) of casein micelles induced using recombinant alpha-S1-casein (dephosphorylated) and kappa casein. Alpha-S1-casein is kept constant, whereas kappa casein is increased 2-fold. Error bars represent standard deviation on triplicate sample.

FIG. 19 shows wet yield (in mg) of cheese made from curds of casein micelles induced using recombinant alpha-S1-casein (dephosphorylated) and kappa casein. Alpha-casein is kept constant, whereas kappa casein is increased 2-fold. Cheese (mozzarella) was made by dipping the curd in hot water, stretching and shaping to a cheese ball.

FIG. 20 shows average micelle diameter (in nm) of casein micelles induced using recombinant alpha-S1-casein (dephosphorylated) and deglycosylated kappa casein. Alpha-S1-casein is kept constant, whereas kappa casein is increased 2-fold. Error bars represent standard deviation on triplicate sample.

FIG. 21 shows wet yield (in mg) of cheese made from curds of casein micelles induced using recombinant alpha-S1-casein (dephosphorylated) and deglycosylated kappa casein. Alpha-casein is kept constant, whereas kappa casein is increased 2-fold. Cheese (mozzarella) was made by dipping the curd in hot water, stretching and shaping to a cheese ball.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Although the dairy industry is worth $330 billion, research needs to be performed for a clean dairy solution using recombinant dairy proteins. As dairy cheese and yogurt are inefficient dairy products, in terms of resources needed per gram as well as being the hardest dairy products to accurately reproduce from just plant-based ingredients, presented herein are methods and compositions of recombinant cheese and recombinant yogurt.

A component that gives dairy cheese or yogurt its unique characteristics is the casein proteins that form micelles in milk. Micelles are protein colloids comprised of four casein proteins (alpha-s1-casein, alpha-s2-casein, beta casein, and kappa casein) that interact with insoluble calcium phosphate at the colloid centre. It is the micelles in milk that attract each other once chymosin is added to milk. This forms the curd, which is then used to make 99% of all cheeses. The current disclosure is based on the discovery that micelles and thereafter cheese can be generated using recombinant alpha and kappa caseins and without the addition of beta casein. In case of yogurt, acidification of the micelle comprising liquid colloid may be performed using a starter culture of bacteria known for yogurt production. The current disclosure also describes micelles and thereafter yogurts that can be generated using recombinant alpha and kappa caseins and without the addition of beta casein.

Recombinant alpha casein and kappa casein may be expressed in a microbial organism, for example, a bacteria such as gram-positive bacteria Lactococcus lactis and Bacillus subtilis, as well as a gram-negative model organism E. coli. These recombinant proteins may be combined with plant-based media (minerals, fats, sugars, and vitamins) to make cheese that behaves, smells, tastes, looks and feels like animal-derived dairy cheese. Recombinant cheese may have no: i) lactose, ii) cholesterol, iii) saturated fats (depending on how it affects the taste and mouthfeel), and iv) whey proteins (often cheese manufacturers cannot fully remove whey from the casein curd in the cheesemaking process).

Methods may include producing recombinant proteins that may require less purification and downstream processing. The bacteria (that are expressing target proteins) may be grown in a rich growth media that may be used in cheese production. The growth media or “serum” may be a plant-based solution, mentioned above, that may be deficient in proteins (as the proteins will be expressed into the media by an engineered bacterial strain).

In some embodiments, the methods include producing recombinant protein in a bacterial host cell, such that such proteins are secreted from the cell into the surrounding media. In some embodiments, the methods include producing recombinant protein in a bacterial host cell, such that such proteins are intracellular. Recombinant protein can then be isolated, purified or partially purified and used in methods for making micelles, liquid colloid, coagulated colloid, curd and cheese.

The fermentation process may be optimized for high protein yields versus body mass, a parameter that can be important for a typical recombinant protein expression via fermentation. The pH may be controlled and/or maintained throughout fermentation so that it does not pass the isoelectric point of proteins expressed. This may be done due to sensitive casein behavior.

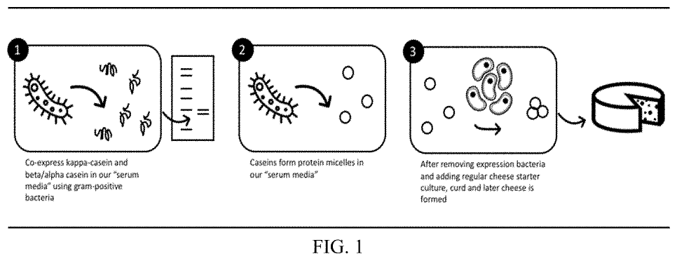

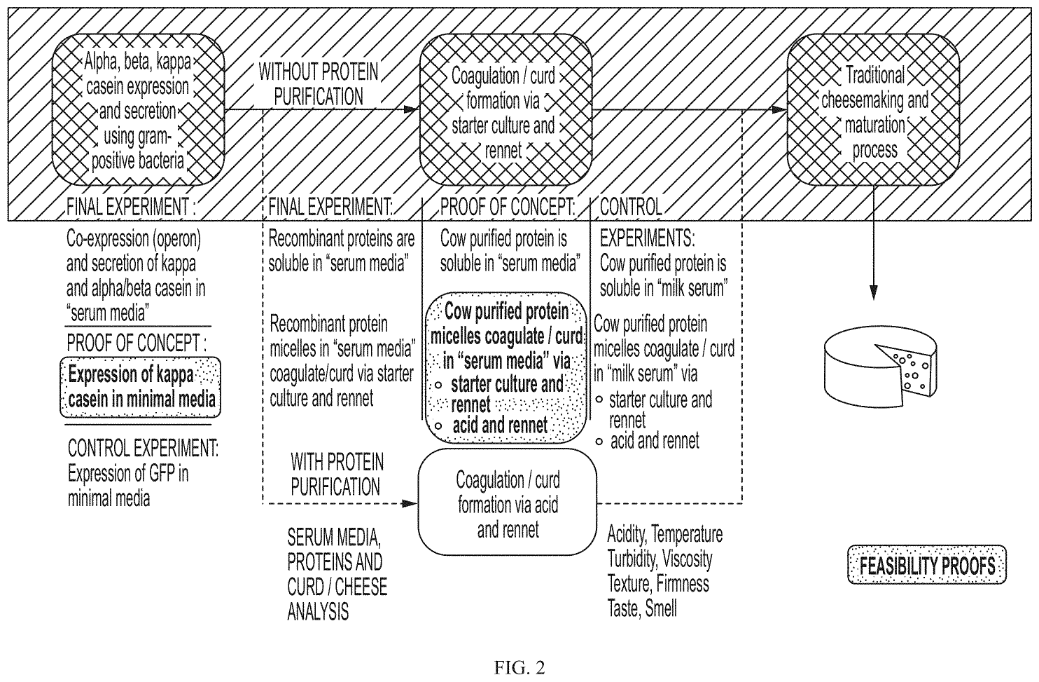

In some embodiments, after reaching an optimal titer, the genetically modified bacteria may be spun out and the supernatant may be harvested, which may be with one or two steps of down-processing to become cheesemaking broth. The main step to cheesemaking broth can be concentrating the solution to reach similar protein concentration to the one found in milk. By this stage, casein micelles may have been formed. After concentration, mesophilic or thermophilic cheesemaking starter culture may be added to ferment the solution until it has reached the right pH for optimal chymosin activity (pH 5.8-6.0 for native micelles, which will likely be different for different micelles). Chymosin may then be added to induce curd formation which can then be made into cheese. FIG. 1 is a basic technical diagram of the production process.

The term “about” as used herein can mean within 1 or more than 1 standard deviation. Alternatively, “about” can mean a range of up to 10%, up to 5%, or up to 1% of a given value. For example, about can mean up to ±10%, ±9%, ±8%, ±7%, ±6%, ±5%, ±4%, ±3%, ±2%, or ±1% of a given value.

The term “dairy protein” as used herein means a protein that has an amino acid sequence derived from a protein found in milk (including variants thereof).

The term “animal-derived” dairy protein as used herein means a protein derived from milk, such as a protein obtained and/or isolated from a milk-producing organism, including but not limited to cow, sheep, goat, human, bison, buffalo, camel and horse. “Animal-derived casein protein” means casein protein obtained and/or isolated from a milk-producing organism.

The term “recombinant dairy protein” as used herein means a protein that is expressed in a heterologous or recombinant organism that has an amino acid sequence derived from a protein found in milk (including variants thereof). “Recombinant casein protein” means a casein produced by a recombinant organism or in a heterologous host cell.

### Compositions and Method of Making Compositions

Cheese compositions herein include coagulated colloids comprising one or more recombinant proteins associated in a micellar form. Micellar forms may be present in a liquid suspension or colloid form. Other components including but not limited to proteins, fats, sugars, minerals, vitamins may be added to the micelles (e.g., to micelles in a liquid colloid). The liquid colloid containing micelles formed with one or more recombinant casein proteins may be treated with acidifying conditions and optionally, coagulating agents such as proteases for curd formation. Thereafter, curds comprising one or more recombinant proteins may then be treated to generate cheese or cheese-like compositions. In case of yogurt formation, the liquid colloid containing micelles formed with one or more recombinant casein proteins may be treated with acidifying conditions such as acidification through a bacterial starter culture.

### I. Micelles and Liquid Colloid

In mammalian milk, casein proteins (alpha-s1-casein, alpha-s2-casein, beta casein, and kappa casein, and a cleaved form of beta casein called gamma casein) and calcium phosphate and citrate form large colloidal particles called casein micelles. The main function of the casein micelle is to provide fluidity to casein molecules and solubilize phosphate and calcium.

Due to the large size of the casein-micelles, which interfere with absolute structure determination, different models of micelle formation have been proposed. Models can be classified into three categories: coat-core model, subunit or sub-micelle model, and internal structure model.

As described herein, casein micelles may be formed with isolated casein proteins, such as recombinantly produced casein protein. Micelles formed from recombinant casein may include either alpha casein, such as alpha-s1-casein and/or alpha-s2-casein, beta casein and/or kappa casein. In some cases, micelles comprise alpha casein and kappa casein. In some cases, micelles comprise alpha casein and kappa casein, and do not contain any beta casein protein.

In some cases, micelles include 2 caseins such as alpha (alpha-S1 or alpha-S2) and kappa casein protein or beta and kappa casein protein. The ratio of alpha or (3-casein protein to κ-casein protein in the micelle may be about 2:1 to 10:1 or about 1:1 to 15:1. The micelle may occupy about 2-6 mL/g and the casein micelle may have an average diameter of 10-400 nm or 10-500 nm.

Two casein proteins forming stable micelles may be co-expressed. This may require engineering and adaptation in form of the exact salt content (calcium, phosphate, potassium, citrate, etc) of the solvent, as well as possibly engineering of casein proteins.

In some embodiments, micelles described herein include micelles formed in a liquid solution. In some embodiments, casein containing micelles are present in a liquid colloid, where the micelles remain dispersed and do not settle out of the liquid solution. In some cases, the liquid colloid includes casein containing micelles and other forms of the caseins such as aggregates and/or monomeric forms of the proteins.

Alpha Casein (α casein): In some embodiments, liquid colloid herein may comprise alpha casein proteins. The alpha casein in liquid colloid may be alpha S1 casein. The alpha casein in liquid colloid may be alpha S2 casein. The alpha casein in liquid colloid may be a combination of alpha S1 and S2 caseins. The alpha casein in liquid colloid may comprise from 0% to 100% of casein. In some instances, a liquid colloid may be produced using only alpha casein, in particular using only alpha S1 casein. Alternatively, in some cases, a liquid colloid may be produced without any alpha casein. In some cases, the alpha casein comprises at least 10%, 20%, 30%, 40%, 50%, 60%, 70%, 80%, 90% or 95% of the casein in liquid colloid. The alpha casein in liquid colloid may comprise from 0% to 100% alpha S1 casein, alpha S2 casein or a combination thereof.

In some cases, casein in liquid colloid comprises of 50% alpha S1 casein to 100% alpha S1 casein. In some cases, liquid colloid comprises alpha casein protein and total casein comprises 100% alpha S1 casein. In some cases, liquid colloid comprises alpha casein protein and total casein comprises at least 50% alpha S1 casein. The alpha casein protein in liquid colloid may comprise from 50% alpha S1 casein to 70% alpha S1 casein, 50% alpha S1 casein to 90% alpha S1 casein, 50% alpha S1 casein to 100% alpha S1 casein, 70% alpha S1 casein to 90% alpha S1 casein, 70% alpha S1 casein to 100% alpha S1 casein, or 90% alpha S1 casein to 100% alpha S1 casein. The alpha casein protein in liquid colloid may comprise about 50% alpha S1 casein, 70% alpha S1 casein, 90% alpha S1 casein, or 100% alpha S1 casein.

In some embodiments, the alpha casein in the liquid colloid is alpha S2 casein. In some cases, casein in liquid colloid comprises of 50% alpha S2 casein to 100% alpha S2 casein. In some cases, liquid colloid comprises alpha casein protein and total casein comprises 100% alpha S2 casein. In some cases, liquid colloid comprises alpha casein protein and total casein comprises at least 50% alpha S2 casein. The alpha casein protein in liquid colloid may comprise from 50% alpha S2 casein to 70% alpha S2 casein, 50% alpha S2 casein to 90% alpha S2 casein, 50% alpha S2 casein to 100% alpha S2 casein, 70% alpha S2 casein to 90% alpha S2 casein, 70% alpha S2 casein to 100% alpha S2 casein, or 90% alpha S2 casein to 100% alpha S2 casein. The alpha casein protein in liquid colloid may comprise 50% alpha S2 casein, 70% alpha S2 casein, 90% alpha S2 casein, or 100% alpha S2 casein.

In some embodiments, the alpha casein in liquid colloid is a mixture of alpha S1 casein and alpha S2 casein. The alpha casein in such liquid colloid may comprise, for example from 1% alpha S2 casein to 99% alpha S2 casein and from 99% alpha S1 casein to 1% alpha S1 casein, respectively. In some embodiments, the alpha casein in liquid colloid is a mixture of alpha S1 casein and alpha S2 casein in ratio of 10:90, 20:80, 30:70, 40:60, 50:50, 60:40, 70:30, 80:20, or 90:10. In some cases, the alpha casein protein in liquid colloid does not include alpha S2 casein. In some cases, the alpha casein protein in liquid colloid does not include alpha S1 casein. In some cases, the alpha casein protein in liquid colloid does not include alpha S2 casein.

The protein content of liquid colloid herein may comprise from 30% to 90% or 50% to 95% alpha casein protein. In some cases, the protein content of liquid colloid may comprise at least 30% alpha casein protein. In some cases, the protein content of liquid colloid may comprise at least 50% alpha casein protein. In some cases, the protein content of liquid colloid may comprise at least 90% or at least 95% alpha casein protein. The protein content of liquid colloid may comprise from 30% to 35%, 30% to 40%, 30% to 50%, 30% to 55%, 30% to 70%, 30% to 75%, 30% to 80%, 30% to 85%, 30% to 90%, 35% to 40%, 35% to 50%, 35% to 55%, 35% to 70%, 35% to 75%, 35% to 80%, 35% to 85%, 35% to 90%, 40% to 50%, 40% to 55%, 40% to 70%, 40% to 75%, 40% to 80%, 40% to 85%, 40% to 90%, 50% to 55%, 50% to 70%, 50% to 75%, 50% to 80%, 50% to 85%, 50% to 90%, 55% to 70%, 55% to 75%, 55% to 80%, 55% to 85%, 55% to 90%, 70% to 75%, 70% to 80%, 70% to 85%, 70% to 90%, 75% to 80%, 75% to 85%, 75% to 90%, 80% to 85%, 80% to 90%, 85% to 90% or 90 to 95% alpha casein protein. The protein content of liquid colloid may comprise 30%, 35%, 40%, 50%, 55%, 70%, 75%, 80%, 85%, 90% or 95% alpha casein protein. The protein content of liquid colloid may comprise at least 30%, 35%, 40%, 50%, 55%, 70%, 75%, 80% 85% or 90% alpha casein protein. The protein content of liquid colloid may comprise at most 40%, 50%, 55%, 70%, 75%, 80%, 85%, 90% or 95% alpha casein protein.

The alpha casein protein (comprising both S1 and/or S2 caseins) may be produced recombinantly. In some cases, liquid colloid may comprise only recombinantly produced alpha casein protein. In certain cases, liquid colloid may comprise substantially only recombinantly produced alpha casein protein. For instance, alpha casein proteins may be 90%, 92%, 95%, 97%, 99% recombinant alpha casein. Alternatively, liquid colloid may comprise a mixture of recombinantly produced and animal-derived alpha casein proteins.

Depending on the host organism used to express the alpha casein, the alpha casein proteins may have a glycosylation or phosphorylation pattern (post-translational modifications) different from animal-derived alpha casein proteins. In some cases, the alpha casein protein comprises no post translational modifications (PTMs). In some cases, the alpha casein protein comprises substantially reduced PTMs. As used herein, substantially reduced PTMs means at least 50% reduction of one or more types of PTMs as compared to the amount of PTMs in an animal-derived alpha casein protein. For instance, alpha casein proteins may be 50%, 55%, 60%, 65%, 70%, 75%, 80%, 85%, 90%, 92%, 95%, 97%, 99% less post-translationally modified as compared to animal-derived alpha casein. Alternatively, the alpha casein protein may comprise PTMs comparable to animal-derived alpha casein PTMs.

The PTMs in the alpha casein protein may be modified chemically or enzymatically. In some cases, the alpha casein protein comprises substantially reduced or no PTMs without chemical or enzymatic treatment. Liquid colloid may be generated using alpha casein protein with reduced or no PTMs, wherein the lack of PTMs is not due to chemical or enzymatic treatments of the protein, such as producing an alpha casein protein through recombinant production where the recombinant protein lacks PTMs.

The phosphorylation in the alpha casein protein may be modified chemically or enzymatically. In some cases, the alpha casein protein comprises substantially reduced or no phosphorylation without chemical or enzymatic treatment. For instance, alpha casein proteins may be 50%, 55%, 60%, 65%, 70%, 75%, 80%, 85%, 90%, 92%, 95%, 97%, 99% less phosphorylated as compared to animal-derived alpha casein. Liquid colloid may be generated using alpha casein protein with reduced or no phosphorylation, wherein the lack of phosphorylation is not due to chemical or enzymatic treatments, such as where recombinant production provides alpha casein protein with reduced or no phosphorylation.

Beta Casein (β casein): In some embodiments, liquid colloid herein comprises a significantly less amount of beta casein protein as compared to an animal-derived micelle (or animal derived liquid colloid). Liquid colloid described herein may be generated to comprise less than 10% beta casein protein. The protein content of liquid colloid herein may comprise less than 10%, 8%, 5%, 3%, 2%, 1% or 0.5% beta casein protein. In preferred embodiments, the liquid colloid described herein do not include any beta casein protein.

Kappa Casein (κ casein): In some embodiments, liquid colloid herein may comprise kappa casein proteins. The protein content of liquid colloid may comprise from 0% to 100% kappa casein protein. The protein content of liquid colloid may comprise at least 1% kappa casein protein. The protein content of liquid colloid may comprise 100% or at most 50% or at most 30% kappa casein protein. Liquid colloid may comprise from 1% to 5%, 1% to 7%, 1% to 10%, 1% to 12%, 1% to 15%, 1% to 18%, 1% to 20%, 1% to 25%, 1% to 30%, 5% to 7%, 5% to 10%, 5% to 12%, 5% to 15%, 5% to 18%, 5% to 20%, 5% to 25%, 5% to 30%, 7% to 10%, 7% to 12%, 7% to 15%, 7% to 18%, 7% to 20%, 7% to 25%, 7% to 30%, 10% to 12%, 10% to 15%, 10% to 18%, 10% to 20%, 10% to 25%, 10% to 30%, 12% to 15%, 12% to 18%, 12% to 20%, 12% to 25%, 12% to 30%, 15% to 18%, 15% to 20%, 15% to 25%, 15% to 30%, 18% to 20%, 18% to 25%, 18% to 30%, 20% to 25%, 20% to 30%, 25% to 30%, 30% to 35%, 35% to 40%, 40 to 45% or 45% to 50% kappa casein protein. The protein content of liquid colloid may comprise 1%, 5%, 7%, 10%, 12%, 15%, 18%, 20%, 25%, 30%, 35%, 40%, 45% or 50%, 60%, 70%, 80%, 90%, or 100% kappa casein protein. The protein content of liquid colloid may comprise at least 1%, 5%, 7%, 10%, 12%, 15%, 18%, 20%, 25%, 30%, 35%, 40% or 45% kappa casein protein. The protein content of liquid colloid may comprise at most 5%, 7%, 10%, 12%, 15%, 18%, 20%, 25%, 30%, 35%, 40%, 45% or 50% kappa casein protein. In some instances, a liquid colloid may be produced using only kappa casein. Alternatively, in some cases, a liquid colloid may be produced without any kappa casein.

The kappa casein protein may be produced recombinantly. In some cases, liquid colloid may comprise only recombinantly produced kappa casein protein. In certain cases, liquid colloid may comprise substantially only recombinantly produced kappa casein protein. In some cases, kappa casein proteins may be 90%, 92%, 95%, 97%, 99% recombinant kappa casein. Alternatively, liquid colloid may comprise a mixture of recombinantly produced and animal-derived kappa casein proteins.

Depending on the host organism used to express the kappa casein, the kappa casein proteins may have a posttranslational modification, such as glycosylation or phosphorylation pattern different from animal-derived kappa casein protein. In some cases, the kappa casein protein in the composition herein comprises no post translational modifications (PTMs). In some cases, the kappa casein protein comprises substantially reduced PTMs. As used herein, substantially reduced PTMs means at least 50% reduction of one or more types of PTMs as compared to the amount of PTMs in an animal-derived kappa casein protein. For instance, kappa casein proteins may be 50%, 55%, 60%, 65%, 70%, 75%, 80%, 85%, 90%, 92%, 95%, 97%, 99% less post-translationally modified as compared to animal-derived kappa casein. Alternatively, the kappa casein protein may comprise PTMs comparable to animal-derived kappa casein PTMs.

The PTMs in the kappa casein protein may be modified chemically or enzymatically. In some cases, the kappa casein protein comprises substantially reduced or no PTMs without chemical or enzymatic treatment. Liquid colloid may be generated using kappa casein protein with reduced or no PTMs, wherein the lack of or reduction of PTMs is not due to chemical or enzymatic treatments, such as by producing recombinant kappa protein in a host where the kappa casein protein is not post-translationally modified or the level of PTMs is substantially reduced.

The glycosylation in the kappa casein protein may be modified chemically or enzymatically. In some cases, the kappa casein protein comprises substantially reduced or no glycosylation without chemical or enzymatic treatment. For instance, kappa casein proteins may be 50%, 55%, 60%, 65%, 70%, 75%, 80%, 85%, 90%, 92%, 95%, 97%, 99% less glycosylated as compared to animal-derived kappa casein. Liquid colloid may be generated using kappa casein protein with reduced or no glycosylation, wherein the lack of glycosylation is not due to chemical or enzymatic treatments post recombinant production.

The phosphorylation in the kappa casein protein may be modified chemically or enzymatically. In some cases, the kappa casein protein comprises substantially reduced or no phosphorylation without chemical or enzymatic treatment. For instance, kappa casein proteins may be 50%, 55%, 60%, 65%, 70%, 75%, 80%, 85%, 90%, 92%, 95%, 97%, 99% less phosphorylated as compared to animal-derived kappa casein. Liquid colloid may be generated using kappa casein protein with reduced or no phosphorylation, wherein the lack of phosphorylation is not due to chemical or enzymatic treatments, such as by producing recombinant kappa protein in a host where the kappa casein protein is not post-translationally modified or the level of PTMs is substantially reduced.

The protein content of a liquid colloid may comprise from about 5% kappa and about 95% alpha casein proteins to about 50% kappa and about 50% alpha casein proteins. The protein content of liquid colloid may comprise about 6% kappa and about 94% alpha, about 5% kappa and about 95% alpha about 7% kappa and about 93% alpha, about 10% kappa and about 90%, alpha, about 12% kappa and about 88% alpha, about 15% kappa and about 85% alpha, about 17% kappa and about 83% alpha, about 20% kappa and about 80% alpha, about 25% kappa and about 75% alpha, about 30% kappa and about 70% alpha casein proteins, about 35% kappa and about 65% alpha, about 40% kappa and about 60% alpha, about 45% kappa and about 55% alpha or about 50% kappa and about 50% alpha.

The ratio of alpha casein protein to kappa casein protein in liquid colloid may be from about 1:1 to about 15:1. The ratio of alpha casein protein to kappa casein protein in liquid colloid may be 1:1, 2:1 to 4:1, 2:1 to 6:1, 2:1 to 8:1, 2:1 to 10:1, 2:1 to 12:1, 2:1 to 14:1, 2:1 to 15:1, 4:1 to 6:1, 4:1 to 8:1, 4:1 to 10:1, 4:1 to 12:1, 4:1 to 14:1, 4:1 to 15:1, 6:1 to 8:1, 6:1 to 10:1, 6:1 to 12:1, 6:1 to 14:1, 6:1 to 15:1, 8:1 to 10:1, 8:1 to 12:1, 8:1 to 14:1, 8:1 to 15:1, 10:1 to 12:1, 10:1 to 14:1, 10:1 to 15:1, 12:1 to 14:1, 12:1 to 15:1, or 14:1 to 15:1. The ratio of alpha casein protein to kappa casein protein in liquid colloid may be about 1:1, 2:1, 3:1, 4:1, 5:1, 6:1, 7:1, 8:1, 9:1, 10:1, 11:1, 12:1, 13:1, 14:1 or 15:1.

In some embodiments, liquid colloid comprises alpha and kappa casein proteins and does not include beta casein, and additionally the alpha casein, kappa casein or both alpha and kappa casein lack post-translational modification(s). For example, liquid colloid comprises alpha casein lacking or substantially reduced in phosphorylation (as compared to alpha casein from animal-derived milk) and kappa casein, or comprises alpha casein lacking or substantially reduced in phosphorylation (as compared to alpha casein from animal-derived milk) and kappa casein that lacks or is substantially reduced in glycosylation or phosphorylation or both glycosylation and phosphorylation (as compared to kappa casein from animal-derived milk). In some cases, liquid colloid comprises alpha casein and comprise kappa casein where the kappa casein is lacking or substantially reduced in glycosylation or phosphorylation or both glycosylation and phosphorylation (as compared to kappa casein from animal-derived milk). In some cases, liquid colloid comprises alpha casein, kappa casein or both produced recombinantly in a bacterial host cell and that lack or are substantially reduced in one or more PTMs.

In some embodiments, liquid colloid herein (and products made therefrom) do not include any dairy proteins other than alpha and kappa casein proteins. In some cases, liquid colloid herein (and products made therefrom) do not include any whey proteins. In some embodiments, liquid colloid herein (and products made therefrom) do not include any animal-derived dairy proteins.

Micelle diameters, such as micelles in liquid colloid, herein may be from about 10 nm to about 500 nm. Micelle diameters herein may be at least 10 nm. Micelle diameters herein may be at most 500 nm. Micelle diameters herein may be from 10 nm to 20 nm, 10 nm to 50 nm, 10 nm to 100 nm, 10 nm to 150 nm, 10 nm to 200 nm, 10 nm to 250 nm, 10 nm to 300 nm, 10 nm to 350 nm, 10 nm to 400 nm, 10 nm to 450 nm, 10 nm to 500 nm, 20 nm to 50 nm, 20 nm to 100 nm, 20 nm to 150 nm, 20 nm to 200 nm, 20 nm to 250 nm, 20 nm to 300 nm, 20 nm to 350 nm, 20 nm to 400 nm, 20 nm to 450 nm, 20 nm to 500 nm, 50 nm to 100 nm, 50 nm to 150 nm, 50 nm to 200 nm, 50 nm to 250 nm, 50 nm to 300 nm, 50 nm to 350 nm, 50 nm to 400 nm, 50 nm to 450 nm, 50 nm to 500 nm, 100 nm to 150 nm, 100 nm to 200 nm, 100 nm to 250 nm, 100 nm to 300 nm, 100 nm to 350 nm, 100 nm to 400 nm, 100 nm to 450 nm, 100 nm to 500 nm, 150 nm to 200 nm, 150 nm to 250 nm, 150 nm to 300 nm, 150 nm to 350 nm, 150 nm to 400 nm, 150 nm to 450 nm, 150 nm to 500 nm, 200 nm to 250 nm, 200 nm to 300 nm, 200 nm to 350 nm, 200 nm to 400 nm, 200 nm to 450 nm, 200 nm to 500 nm, 250 nm to 300 nm, 250 nm to 350 nm, 250 nm to 400 nm, 250 nm to 450 nm, 250 nm to 500 nm, 300 nm to 350 nm, 300 nm to 400 nm, 300 nm to 450 nm, 300 nm to 500 nm, 350 nm to 400 nm, 350 nm to 450 nm, 350 nm to 500 nm, 400 nm to 450 nm, 400 nm to 500 nm, or 450 nm to 500 nm. Micelle diameters herein may be about 10 nm, about 20 nm, about 50 nm, about 100 nm, about 150 nm, about 200 nm, about 250 nm, about 300 nm, about 350 nm, about 400 nm, about 450 nm, or about 500 nm. Micelle diameters herein may be at least 10 nm, 20 nm, 50 nm, 100 nm, 150 nm, 200 nm, 250 nm, 300 nm, 350 nm, 400 nm or 450 nm. Micelle diameters herein may be at most 20 nm, 50 nm, 100 nm, 150 nm, 200 nm, 250 nm, 300 nm, 350 nm, 400 nm, 450 nm or 500 nm.

Salts: A casein mixture in a liquid colloid may comprise alpha, beta and/or kappa casein proteins as described elsewhere herein. In some embodiments, liquid colloid includes alpha casein and kappa casein, but does not include beta casein. Micelle formation in liquid colloid herein may comprise addition of various salts to a solution comprising a casein mixture. Salts that may be added to a casein mixture may include calcium, phosphorous, citrate, potassium, sodium and/or chloride salts. In some cases, salt is comprised within the micelles. In some cases, salt is comprised in the liquid colloid such that a proportion of salt is comprised in the micelles and another portion of salt is in solution (e.g., “outside” the micelles).

Liquid colloid containing casein micelles may comprise a calcium salt. The calcium salt may be selected from calcium chloride, calcium carbonate, calcium citrate, calcium glubionate, calcium lactate, calcium gluconate, calcium acetate, equivalents thereof and/or combinations thereof. The concentration of a calcium salt in liquid colloid may be from about 10 mM to about 55 mM. The concentration of a calcium salt in liquid colloid may be at least 10 mM. The concentration of a calcium salt in liquid colloid may be at most 50 mM. In some embodiments, the concentration of a calcium salt in liquid colloid may be 28 mM or no more than 28 mM or may be 55 mM or no more than 55 mM. The concentration of a calcium salt in liquid colloid may be 10 mM to 15 mM, 10 mM to 20 mM, 10 mM to 25 mM, 10 mM to 30 mM, 10 mM to 35 mM, 10 mM to 40 mM, 10 mM to 45 mM, 10 mM to 50 mM, 10 mM to 55 mM, 15 mM to 20 mM, 15 mM to 25 mM, 15 mM to 30 mM, 15 mM to 35 mM, 15 mM to 40 mM, 15 mM to 45 mM, 15 mM to 50 mM, 15 mM to 55 mM, 20 mM to 25 mM, 20 mM to 30 mM, 20 mM to 35 mM, 20 mM to 40 mM, 20 mM to 45 mM, 20 mM to 50 mM, 20 mM to 55 mM, 25 mM to 30 mM, 25 mM to 35 mM, 25 mM to 40 mM, 25 mM to 45 mM, 25 mM to 50 mM, 25 mM to 55 mM, 30 mM to 35 mM, 30 mM to 40 mM, 30 mM to 45 mM, 30 mM to 50 mM, 30 mM to 55 mM, 35 mM to 40 mM, 35 mM to 45 mM, 35 mM to 50 mM, 35 mM to 55 mM, 40 mM to 45 mM, 40 mM to 50 mM, 40 mM to 55 mM, 45 mM to 50 mM, 45 mM to 55 mM, or 50 mM to 55 mM. The concentration of a calcium salt in liquid colloid may be 10 mM, 15 mM, 20 mM, 25 mM, 30 mM, 35 mM, 40 mM, 45 mM, 50 mM, or 55 mM. The concentration of a calcium salt in liquid colloid may be at least 10 mM, 15 mM, 20 mM, 25 mM, 30 mM, 35 mM, 40 mM, 45 mM or 50 mM. The concentration of a calcium salt in liquid colloid may be at most 15 mM, 20 mM, 25 mM, 30 mM, 35 mM, 40 mM, 45 mM, 50 mM or 55 mM.

Liquid colloid containing casein micelles may comprise a phosphate salt. The phosphate salt may be selected from orthophosphates such as monosodium (dihydrogen) phosphate, disodium phosphate, trisodium phosphate, monopotassium (dihydrogen) phosphate, dipotassium phosphate, tripotassium phosphate; pyrophosphates such as disodium or dipotassium pyrophosphate, trisodium or tripotassium pyrophosphate, tetrasodium or tetrapotassium pyrophosphate; polyphosphates such as pent sodium or potassium tripolyphosphate, sodium or potassium tetrapolyphosphate, sodium or potassium hexametaphosphate. The concentration of a phosphate salt in liquid colloid may be from about 8 mM to about 45 mM. The concentration of a phosphate salt in liquid colloid may be at least 8 mM. The concentration of a phosphate salt in liquid colloid may be at most 25 mM or at most 30 mM or at most 40 mM or at most 45 mM. The concentration of a phosphate salt in liquid colloid may be 8 mM to 10 mM, 8 mM to 15 mM, 8 mM to 20 mM, 8 mM to 25 mM, 8 mM to 30 mM, 8 mM to 35 mM, 8 mM to 40 mM, 8 mM to 45 mM, 10 mM to 15 mM, 10 mM to 20 mM, 10 mM to 25 mM, 10 mM to 30 mM, 10 mM to 35 mM, 10 mM to 40 mM, 10 mM to 45 mM, 15 mM to 20 mM, 15 mM to 25 mM, 15 mM to 30 mM, 15 mM to 35 mM, 15 mM to 40 mM, 15 mM to 45 mM, 20 mM to 25 mM, 20 mM to 30 mM, 20 mM to 35 mM, 20 mM to 40 mM, 20 mM to 45 mM, 25 mM to 30 mM, 25 mM to 35 mM, 25 mM to 40 mM, 25 mM to 45 mM, 30 mM to 35 mM, 30 mM to 40 mM, 30 mM to 45 mM, 35 mM to 40 mM, 35 mM to 45 mM, or 40 mM to 45 mM. The concentration of a phosphate salt in liquid colloid may be about 8 mM, 10 mM, 15 mM, 20 mM, 25 mM, 30 mM, 35 mM, 40 mM, or 45 mM. The concentration of a phosphate salt in liquid colloid may be at least 8 mM, 10 mM, 15 mM, 20 mM, 25 mM, 30 mM, 35 mM or 40 mM. The concentration of a phosphate salt in liquid colloid may be at most 10 mM, 15 mM, 20 mM, 25 mM, 30 mM, 35 mM, 40 mM or 45 mM.

Liquid colloid containing casein micelles may comprise a citrate salt. The citrate salt may be selected from calcium citrate, potassium citrate, sodium citrate, trisodium citrate, tripotassium citrate or equivalents thereof. The concentration of a citrate salt in liquid colloid may be from about 2 mM to about 20 mM. The concentration of a citrate salt in liquid colloid may be at least 2 mM. The concentration of a citrate salt in liquid colloid may be at most 15 mM or at most 20 mM. The concentration of a citrate salt in liquid colloid may be 2 mM to 4 mM, 2 mM to 6 mM, 2 mM to 8 mM, 2 mM to 10 mM, 2 mM to 12 mM, 2 mM to 14 mM, 2 mM to 16 mM, 2 mM to 18 mM, 2 mM to 20 mM, 4 mM to 6 mM, 4 mM to 8 mM, 4 mM to 10 mM, 4 mM to 12 mM, 4 mM to 14 mM, 4 mM to 16 mM, 4 mM to 18 mM, 4 mM to 20 mM, 6 mM to 8 mM, 6 mM to 10 mM, 6 mM to 12 mM, 6 mM to 14 mM, 6 mM to 16 mM, 6 mM to 18 mM, 6 mM to 20 mM, 8 mM to 10 mM, 8 mM to 12 mM, 8 mM to 14 mM, 8 mM to 16 mM, 8 mM to 18 mM, 8 mM to 20 mM, 10 mM to 12 mM, 10 mM to 14 mM, 10 mM to 16 mM, 10 mM to 18 mM, 10 mM to 20 mM, 12 mM to 14 mM, 12 mM to 16 mM, 12 mM to 18 mM, 12 mM to 20 mM, 14 mM to 16 mM, 14 mM to 18 mM, 14 mM to 20 mM, 16 mM to 18 mM, 16 mM to 20 mM, or 18 mM to 20 mM. The concentration of a citrate salt in liquid colloid may be 2 mM, 4 mM, 6 mM, 8 mM, 10 mM, 12 mM, 14 mM, 16 mM, 18 mM, or 20 mM. The concentration of a citrate salt in liquid colloid may be at least 2 mM, 4 mM, 6 mM, 8 mM, 10 mM, 12 mM, 14 mM, 16 mM or 18 mM. The concentration of a citrate salt in liquid colloid may be at most 4 mM, 6 mM, 8 mM, 10 mM, 12 mM, 14 mM, 16 mM, 18 mM, or 20 mM.

Liquid colloid containing casein micelles may comprise a combination of salts. In some embodiments, the liquid colloid comprises calcium, phosphate and citrate salts. In some cases, a ratio of calcium, phosphate and citrate salt in liquid colloid may be from 3:2:1 to about 6:4:1. A ratio of calcium, phosphate and citrate salt in liquid colloid may be about 3:1:1, 3:2:1, 3:3:1, 4:2:1, 4:3:1, 4:4:1, 5:2:1, 5:2:2, 5:3:1, 5:4:1, 5:5:1, 5:3:2, 5:4:2, 6:1:1, 6:2:1, 6:3:1 or 6:4:1.

Micelle formation in liquid colloid may require solubilization of casein proteins in a solvent such as water. Salts may be added after the solubilization of casein proteins in a solvent. Alternatively, salts and casein proteins may be added to the solution simultaneously. Salts may be added more than once during micelle formation. For instance, calcium salts, phosphate salts and citrate salts may be added at regular intervals or in a continuous titration process and mixed in a solution comprising casein proteins until a micellar liquid colloid of desired quality is generated. In one example, salts may be added at regular interval till the colloid reaches a desired absorbance. Different salts may be added at different times during the micelle formation process. For instance, calcium salts may be added before the addition of phosphate and citrate salts, or citrate salts may be added before the addition of calcium and phosphate salts, or phosphate salts might be added before the addition of calcium and citrate salts.

Additional components may be added to liquid colloid such that the liquid colloid is then milk-like and used for curd and/or cheese or yogurt formation. In some embodiments, fat is added to liquid colloid. In some cases, fats may be essentially free of animal-derived fats. Fats used herein may include plant-based fats such as canola oil, sunflower oil, coconut oil or combinations thereof. The concentration of fats may be about 0% to about 5% in the liquid colloid. The concentration of fats may be at least 0.5% or about 1%. The concentration of fats may be at most 5%. The concentration of fats may be about 0%, 0.1%, 0.5%, 1%, 2%, 3%, 4% or 5%. The concentration of fats may be from 0 to 0.5%, 0.5% to 1%, 1% to 3%, 1% to 4%, or 1% to 5%. The concentration of fats may be at most 2%, 3%, 4%, or 5%.

Liquid colloid as described herein may further comprise sugars. Sugars used herein may include plant-based dissacharides and/or oligosaccharides. Examples of sugars include sucrose, glucose, fructose, galactose, lactose, maltose, mannose, allulose, tagatose, xylose, and arabinose.

Liquid colloid with additional components may be generated by mixing different components at a temperature from 30° C. to 45° C. For instance, liquid colloid with one or more recombinant proteins (such as a combination of alpha and kappa casein) may be mixed with fats and/or sugars at a temperature of about 30° C., 32° C., 35° C., 37° C., 40° C., 42° C. or 45° C.

### II. Curd/Cheese, Yogurt Formation and Components

Micelles such as micelles of alpha and kappa casein, may be present in a liquid colloid, where a substantial portion of the micelles remain in suspension in the liquid. In some embodiments, the liquid colloid is treated to form a coagulated colloid. In some cases, the treatment is a reduction of pH of the liquid colloid such as by adding acid or acidifying with a microorganism, to generate coagulated colloid.

Fats may be added to liquid colloid for the generation of a coagulated colloid or curds such that in a final cheese product the concentration of fat is between about 0% to about 50%, typically more than 0%. For example, the concentration of fat in the cheese product made from liquid colloid is about 5%, 10%, 15%, 20%, 25%, 30%, 35%, 40%, 45% or 50%. The concentration of fat in the cheese product made from liquid colloid may be between 1% to 50%. The concentration of fat in the cheese product made from liquid colloid may be at least 1%. The concentration of fat in the cheese product made from liquid colloid may be at most 50%. The concentration of fat in the cheese product made from liquid colloid may be 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50%. The concentration of fat in the cheese product made from liquid colloid may be 1%, 5%, 10%, 15%, 20%, 25%, 30%, 35%, 40%, 45%, or 50%. The concentration of fat in the cheese product made from liquid colloid may be at least 1%, 5%, 10%, 15%, 20%, 25%, 30%, 35%, 40%, 45%, or 50%. The concentration of fat in the cheese/yogurt product made from liquid colloid may be at most 1%, 5%, 10%, 15%, 20%, 25%, 30%, 35%, 40% or 45%.

Fats may be emulsified into liquid colloid (e.g. comprising micelles formed with alpha and kappa casein and salt) using sonication or high-pressure homogenization process. An emulsifier such as soy lecithin or xanthan gum may be used to secure a stable emulsion.

Coagulated colloid may be generated at a final pH of about 4 to about 6. Coagulated colloid may be generated at a pH of about 4 to about 6. Coagulated colloid may be generated at a final pH of at least 4. Coagulated colloid may be generated at a final pH of at most 6. Coagulated colloid may be generated at a final pH of 4 to 4.5, 4 to 5, 4 to 5.1, 4 to 5.2, 4 to 5.5, 4 to 6, 4.5 to 5, 4.5 to 5.1, 4.5 to 5.2, 4.5 to 5.5, 4.5 to 6, 5 to 5.1, 5 to 5.2, 5 to 5.5, 5 to 6, 5.1 to 5.2, 5.1 to 5.5, 5.1 to 6, 5.2 to 5.5, 5.2 to 6, or 5.5 to 6. Coagulated colloid may be generated at a final pH of about 4, about 4.5, about 5, about 5.1, about 5.2, about 5.5, or about 6. Coagulated colloid may be generated at a final pH of at least 4, 4.5, 5, 5.1, 5.2 or 5.5. Coagulated colloid may be generated at a final pH of at most 4.5, 5, 5.1, 5.2, 5.5, or 6. Treatments for reducing pH of liquid colloid and achieving a final pH or final pH range described herein may include the addition of an acid such as citric acid, lactic acid, or vinegar (acetic acid). Treatments for reducing pH of liquid colloid and achieving a final pH or final pH range described herein may include the addition of an acidifying microorganism such as lactic acid bacteria. Exemplary acidifying microorganisms include Lactococci, Streptococci, Lactobacilli and mixtures of thereof. In some cases, both acid and an acidifying microorganism are added to the liquid colloid to create a coagulated colloid. In some cases, aging and ripening microorganisms (such as bacteria or fungi) are also added in this step.

In some cases, following acidification, a renneting agent may be added to form a renneted curd (coagulated curd matrix), which may then be used to make cheese. Micelles in a liquid colloid, such as milk and also the liquid colloid described herein, are stable and repel each other in colloidal suspension. In presence of renneting agents or milk-clotting enzymes, and when acidified, micelles are destabilized and attract each other, and thus coagulate. In presence of renneting agents or milk-clotting enzymes, cross-linked coagulated curd matrix is formed. Renneting agents used for curd formation may include chymosin, pepsin A, mucorpepsin, enthothiapepsin or equivalents thereof. Renneting agents may be derived from plants, dairy products or recombinantly.

In some embodiments, renneted curd is further treated to create a cheese or cheese-like product. In some cases, such as a mozzarella product, the renneted curd may be heated and stretched. In other embodiments, the renneted curd is aged, such as for brie, camembert, feta, halloumi, gouda, edam, cheddar, manchego, swiss, colby, muenster, blue cheese or parmesan type cheese or cheese-like product.

In some embodiments, coagulated colloid or renneted curd may be treated with hot water for the formation of cheese, such as for mozzarella-type cheese. Hot water treatment may be performed at a temperature of about 50° C. to about 90° C. Hot water treatment may be performed at a temperature of at least 55° C. Hot water treatment may be performed at a temperature of at most 75° C. Hot water treatment may be performed at a temperature of 50° C. to 55° C., 55° C. to 60° C., 55° C. to 65° C., 55° C. to 70° C., 55° C. to 75° C., 60° C. to 65° C., 60° C. to 70° C., 60° C. to 75° C., 65° C. to 70° C., 65° C. to 75° C., 70° C. to 75° C., 75° C. to 80° C., 80° C. to 85° C., or 85° C. to 90° C. Hot water treatment may be performed at a temperature of about 50° C., about 55° C., about 60° C., about 65° C., about 70° C., about 75° C., about 80° C., about 85° C. or about 90° C. Hot water treatment may be performed at a temperature of at least 50° C., 55° C., 60° C., 65° C., 70° C., 75° C., 80° C., or 85° C. Hot water treatment may be performed at a temperature of at most 55° C., 60° C., 65° C., 70° C., 75° C., 80° C., 85° C. or 90° C. In some cases, after hot water treatment, the product is stretched into a cheese. In some cases, the cheese is a mozzarella-like cheese.

Cheese compositions formed using the methods described herein may not comprise any animal-derived components. Cheese compositions formed using the methods described herein may not comprise any animal-derived dairy-based components, such as animal-derived dairy proteins. Cheese compositions formed using the methods described herein may not comprise any whey proteins. Cheese compositions formed using the methods described herein may not comprise any beta casein protein. Cheese compositions described herein may be pasta-filata like cheese such as mozzarella cheese. Soft cheeses such as paneer, cream cheese or cottage cheese may also be formed using the methods described herein. Other types of cheese such as aged and ripened cheeses may also be formed using the methods described herein, such as brie, camembert, feta, halloumi, gouda, edam, cheddar, manchego, swiss, colby, muenster, blue cheese and parmesan.

The texture of a cheese made by methods described herein may be comparable to the texture of a similar type of cheese made using animal-derived dairy derived proteins, such as cheese made from animal milk. Texture of a cheese may be tested using a trained panel of human subjects or machines such as a texture analyzer.

The taste of a cheese made by methods described herein may be comparable to a similar type of cheese made using animal-derived dairy proteins. Taste of a cheese may be tested using a trained panel of human subjects.

Cheese compositions described herein may have a browning ability which is comparable to a similar type of cheese made using animal-derived dairy proteins. Cheese compositions described herein may have a melting ability which is comparable to a similar type of cheese made using animal-derived dairy proteins.

In some embodiments, the liquid colloid may be used for yogurt formation. In some cases, for yogurt production, the liquid colloid may be heat treated. The heat treatment may include treating the liquid colloid at a temperature of about 75° C., 80° C., 85° C., 87° C., 90° C., 92° C., 95° C., or 100° C. The heat treatment may be followed with a cooling step of the liquid colloid.

In some cases, for instance, in yogurt production, a bacterial culture may be used as a starter culture. Starter bacterial cultures used for yogurt production may be any bacterial cultures known in the art. For instance, bacteria known for yogurt generation such as Lactobacillus delbrueckii subsp. bulgaricus, Streptococcus thermophilus, other lactobacilli and bifidobacteria sp. bacteria may be cultured and added to the liquid colloid comprising the one or more recombinant proteins. The bacterial starter culture may be used for the acidification of the liquid colloid. Acidification of a liquid colloid may be continued until a desired consistency of the colloid is achieved. For instance, bacterial acidification may be continued until a desired consistency is reached for the liquid colloid. Bacterial acidification of the liquid colloid may lead to the formation of a coagulated liquid colloid which has a yogurt-like consistency.

Bacterial acidification of the liquid colloid in yogurt production may be performed at a temperature between 30° C. to 55° C. In some cases, bacterial acidification of the liquid colloid may be performed at temperature of at least 30° C. Bacterial acidification of the liquid colloid may be performed at temperature of at most 55° C. Bacterial acidification of the liquid colloid may be performed at temperature of 30° C. to 35° C., 30° C. to 40° C., 30° C. to 45° C., 30° C. to 50° C., 30° C. to 55° C., 35° C. to 40° C., 35° C. to 45° C., 35° C. to 50° C., 35° C. to 55° C., 40° C. to 45° C., 40° C. to 50° C., 40° C. to 55° C., 45° C. to 50° C., 45° C. to 55° C., or 50° C. to 55° C. Bacterial acidification of the liquid colloid may be performed at temperature of about 30° C., 35° C., 40° C., 45° C., 50° C., or 55° C. Bacterial acidification of the liquid colloid may be performed at temperature of at least 30° C., 35° C., 40° C., 45° C. or 50° C. Bacterial acidification of the liquid colloid may be performed at temperature of at most 35° C., 40° C., 45° C., 50° C., or 55° C. In some cases, bacterial acidification may be performed at a temperature between 30° C. to 55° C. for at least 1 hour. In some cases, bacterial acidification may be performed at a temperature between 30° C. to 55° C. for at least 2 hours, at least 3 hours, at least 4 hours, at least 5 hours at least 6 hours, at least 8 hours, at least 10 hours or at least 12 hours. In some cases, bacterial acidification may be performed at a temperature between 30° C. to 55° C. for at most 1 hour. In some cases, bacterial acidification may be performed at a temperature between 30° C. to 55° C. for at most 2 hours, at most 3 hours, at most 4 hours, at most 5 hours, at most 6 hours, at most 8 hours, at most 10 hours or at most 12 hours.

Alternatively, bacterial acidification may be performed at a lower temperature between 15° C. to 30° C. Bacterial acidification of the liquid colloid may be performed at temperature of at least 15° C. Bacterial acidification of the liquid colloid may be performed at temperature of at most 30° C. Bacterial acidification of the liquid colloid may be performed at temperature of 15° C. to 17° C., 15° C. to 20° C., 15° C. to 22° C., 15° C. to 25° C., 15° C. to 27° C., 15° C. to 30° C., 17° C. to 20° C., 17° C. to 22° C., 17° C. to 25° C., 17° C. to 27° C., 17° C. to 30° C., 20° C. to 22° C., 20° C. to 25° C., 20° C. to 27° C., 20° C. to 30° C., 22° C. to 25° C., 22° C. to 27° C., 22° C. to 30° C., 25° C. to 27° C., 25° C. to 30° C., or 27° C. to 30° C. Bacterial acidification of the liquid colloid may be performed at temperature of about 15° C., 17° C., 20° C., 22° C., 25° C., 27° C., or 30° C. Bacterial acidification of the liquid colloid may be performed at temperature of at least 15° C., 17° C., 20° C., 22° C., 25° C. or 27° C. Bacterial acidification of the liquid colloid may be performed at temperature of at most 17° C., 20° C., 22° C., 25° C., 27° C., or 30° C. In some cases, bacterial acidification may be performed at a temperature between 15° C. to 30° C. for at least 10 hours. In some cases, bacterial acidification may be performed at a temperature between 15° C. to 30° C. for at least 10 hours, at least 12 hours, at least 14 hours, at least 16 hours at least 18 hours, at least 20 hours, at least 22 hours or at least 24 hours. In some cases, bacterial acidification may be performed at a temperature between 15° C. to 30° C. for at most 24 hours. In some cases, bacterial acidification may be performed at a temperature between 15° C. to 30° C. for at most 12 hours, at most 14 hours, at most 16 hours, at most 18 hours, at most 20 hours, at most 22 hours or at most 24 hours.

Similar to cheese formation, a coagulated liquid colloid for yogurt formation may comprise other components such as sugars, fats, stabilizers and flavouring agents.

The concentration of fat in the yogurt product made from liquid colloid may be 0% to 12%. The yogurt product made from liquid colloid may comprise less than 1% fat, or in some cases no fats. The concentration of fat in the yogurt product made from liquid colloid may be at most 12%. The concentration of fat in the cheese product made from liquid colloid may be 1% to 2%, 1% to 5%, 1% to 7%, 1% to 10%, 1% to 12%, 2% to 5%, 2% to 7%, 2% to 10%, 2% to 12%, 5% to 7%, 5% to 10%, 5% to 12%, 7% to 10%, 7% to 12%, or 10% to 12%. The concentration of fat in the cheese product made from liquid colloid may be about 1%, 2%, 5%, 7%, 10%, or 12%. The concentration of fat in the cheese product made from liquid colloid may be at least 1%, 2%, 5%, 7% or 10%. The concentration of fat in the cheese product made from liquid colloid may be at most 2%, 5%, 7%, 10%, or 12%. Fats may be emulsified into liquid colloid (e.g. comprising micelles formed with alpha and kappa casein and salt) using sonication or high-pressure homogenization process. An emulsifier such as soy lecithin or xanthan gum may be used to secure a stable emulsion

The texture of a yogurt made by methods described herein may be comparable to the texture of a similar type of yogurt made using animal-derived dairy derived proteins, such as yogurt made from animal milk. Texture of a yogurt may be tested using a trained panel of human subjects or machines such as a texture analyzer.

The taste of a yogurt made by methods described herein may be comparable to a similar type of yogurt made using animal-derived dairy proteins. Taste of a yogurt may be tested using a trained panel of human subjects.

### Recombinant Expression

One or more proteins used in the formation of cheese compositions may be produced recombinantly. In some cases, alpha S1, alpha S2 and kappa casein are produced recombinantly.

Alpha S1 and/or S2 casein can have an amino acid sequence from any species. For example, recombinant alpha casein may have an amino acid sequence of cow, human, sheep, goat, buffalo, bison, horse or camel alpha casein. Alpha casein nucleotide sequence may be codon-optimized for increased efficiency of production. Exemplary alpha casein protein sequences are provided in Table 1 below. Recombinant alpha casein can be a non-naturally occurring variant of an alpha casein. Such variant can comprise one or more amino acid insertions, deletions, or substitutions relative to a native alpha casein sequence.

Such a variant can have at least 70%, 75%, 80%, 85%, 90%, 95%, 96%, 97%, 98%, or 99% sequence identity to SEQ ID NOs: 1-26. The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

Kappa casein can have an amino acid sequence from any species. For example, recombinant kappa casein may have an amino acid sequence of cow, human, sheep, goat, buffalo, bison, horse, or camel kappa casein. Kappa casein nucleotide sequence may be codon-optimized for increased efficiency of production. Exemplary kappa casein amino acid sequences are provided in Table 1 below. Recombinant kappa casein can be a non-naturally occurring variant of a kappa casein. Such variant can comprise one or more amino acid insertions, deletions, or substitutions relative to a native kappa sequence.

Such a variant can have at least 70%, 75%, 80%, 85%, 90%, 95%, 96%, 97%, 98%, or 99% sequence identity to SEQ ID NOs: 27-40.

A recombinant alpha or kappa casein is recombinantly expressed in a host cell. As used herein, a “host” or “host cell” denotes any protein production host selected or genetically modified to produce a desired product. Exemplary hosts include fungi, such as filamentous fungi, as well as bacteria, yeast, plant, insect, and mammalian cells. In some cases, a bacterial host cell such as Lactococcus lactis, Bacillus subtilis or Escherichia coli may be used to produce alpha and/or kappa casein proteins. Other host cells include bacterial host such as, but not limited to, Lactococci sp., Lactococcus lactis, Bacillus subtilis, Bacillus amyloliquefaciens, Bacillus licheniformis and Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum and Synechocystis sp. 6803.

Alpha and kappa caseins may be produced in the same host cell. Alternatively, alpha and kappa casein may be produced in different host cells. Expression of a target protein can be provided by an expression vector, a plasmid, a nucleic acid integrated into the host genome or other means. For example, a vector for expression can include: (a) a promoter element, (b) a signal peptide, (c) a heterologous casein sequence, and (d) a terminator element. In some cases, the one or more expression vectors described herein do not comprise a protein sequence for beta casein (SEQ ID NOs: 41-42).

Expression vectors that can be used for expression of casein include those containing an expression cassette with elements (a), (b), (c) and (d). In some embodiments, the signal peptide (c) need not be included in the vector. In some cases, a signal peptide may be part of the native signal sequence of the casein protein, for instance, the protein may comprise a native signal sequence as bolded in SEQ ID NOs: 1, 3, 5, 7, 9, 11, 13, 15, 17, 19, 21, 23, 25, 27, 29, 31, 33, 35, 37, 39 or 41. In some cases, the vector comprises a protein sequence as exemplified in SEQ ID NOs: 1, 3, 5, 7, 9, 11, 13, 15, 17, 19, 21, 23, 25, 27, 29, 31, 33, 35, 37, 39 or 41. In some cases, the vector may comprise a mature protein sequence, as exemplified in SEQ ID NOs: 2, 4, 6, 8, 10, 12, 14, 16, 18, 20, 22, 24, 26, 28, 30, 32, 34, 36, 38, 40 with a heterologous signal sequence. In general, the expression cassette is designed to mediate the transcription of the transgene when integrated into the genome of a cognate host microorganism or when present on a plasmid or other replicating vector maintained in a host cell.

To aide in the amplification of the vector prior to transformation into the host microorganism, a replication origin (e) may be contained in the vector. To aide in the selection of microorganism stably transformed with the expression vector, the vector may also include a selection marker (f). The expression vector may also contain a restriction enzyme site (g) that allows for linearization of the expression vector prior to transformation into the host microorganism to facilitate the expression vectors stable integration into the host genome. In some embodiments the expression vector may contain any subset of the elements (b), (e), (f), and (g), including none of elements (b), (e), (f), and (g). Other expression elements and vector element known to one of skill in the art can be used in combination or substituted for the elements described herein.

Gram positive bacteria (such as Lactococcus lactis and Bacillus subtilis) may be used to secrete target proteins into the media, and gram-negative bacteria (such as Escherichia coli) may be used to secrete target proteins into periplasm or into the media. In some embodiments, the bacterially-expressed proteins expressed may not have any post-translational modifications (PTMs), which means they are not glycosylated and/or may not be phosphorylated.

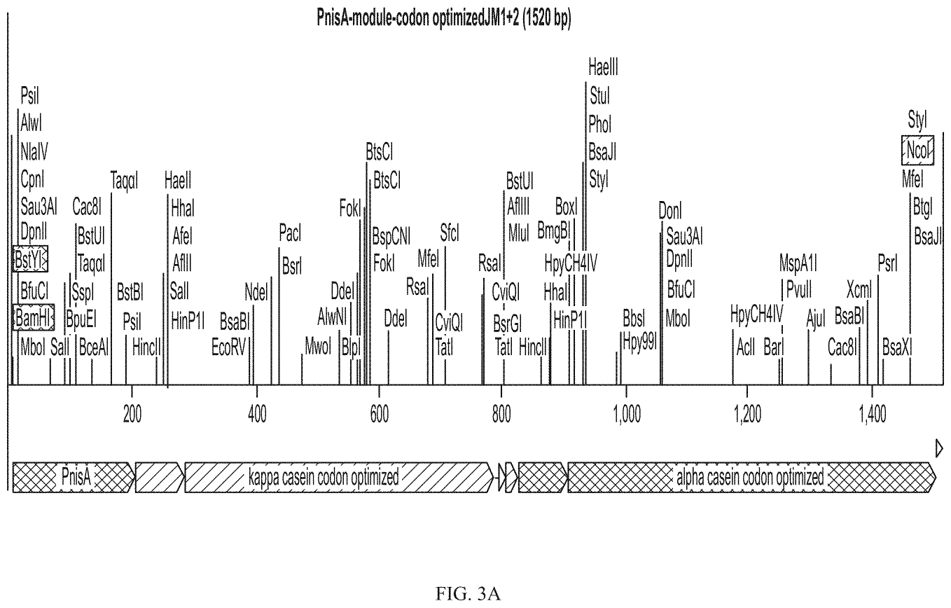

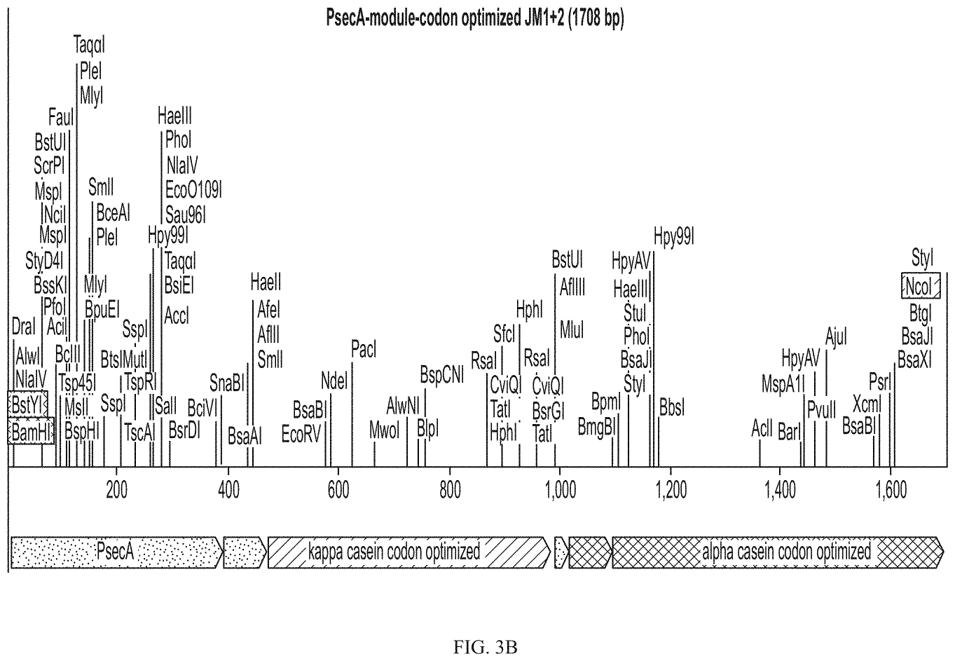

Target casein proteins may be expressed and produced in L. lactis both in a nisin-inducible expression system (regulated by PnisA promoter), lactate-inducible expression system (regulated by P170 promoter) or other similar inducible systems, as well as a constitutively expressed system (regulated by P secA promoter), wherein both are in a food-grade selection strain, such as NZ3900 using vector pNZ8149 (lacF gene supplementation/rescue principle). The secretion of functional proteins may be enabled by the signal peptide of Usp45 (SP(usp45)), the major Sec-dependent protein secreted by L. lactis. For example, alpha-S1-casein and kappa casein may be co-expressed or individually expressed in L. lactis using a synthetic operon, where the gene order is kappa casein-alpha S1 casein, as shown in FIGS. 3A and 3B.

Bacillus subtilis Design

B. subtilis, unlike L. lactis, has multiple intracellular and extracellular proteases, which may interfere with protein expression. In some embodiments, B. subtilis strains are modified to reduce the type and amount of intracellular and/or extracellular proteases, for example strains which have deletions for 7 (K07) and 8 (WB800N) proteases, respectively, may be used.

In order to drive the recombinant protein secretion, the signal peptide of amyQ, alpha-amylase of Clostridium thermocellum may be used. Additionally, native casein signal peptide sequences may be expressed heterologously in B. subtilis. Each casein protein has its own signal peptide sequence and may be used in the system. The signal proteins may be cross-combined with the casein proteins. The pHT01 vector may be used as a transformation and expression shuttle for inducible protein expression in B. subtilis. The vector is based on the strong σ^{A}-dependent promoter preceding the groES-groELoperon of B. subtilis, which has been converted into an efficiently controllable (IPTG-inducible) promoter by addition of the lac operator. pHT01 is an E. coli/B. subtilis shuttle vector that provides ampicillin resistance to E. coli and chloramphenicol resistance to B. subtilis.

Untagged and tagged versions of caseins may be expressed, whereby a small peptide tag such as His or StrepII tag, sequence or fusion protein such as GST, MBP or SUMO is placed N- or C-terminally to casein without the secretion signal peptide. Given secondary structures of kappa, alpha-S1, and alpha S2 casein, tagging may be less disruptive at N-terminal of kappa casein, whereby alpha-S1 casein can likely be tagged at both termini. However, other tags may be used.

TABLE 1
Sequences

| SEQ ID | | |
|---|---|---|
| No. | Name | Sequence |
| 1 | Bovine Alpha-S1 Casein | MKLLILTCLVAVALARPKHPIKHQGLPQEVLNENL |
| | | LRFFVAPFPEVFGKEKVNELSKDIGSESTEDQAMED |
| | | IKQMEAESISSSEEIVPNSVEQKHIQKEDVPSERYLG |
| | | YLEQLLRLKKYKVPQLEIVPNSAEERLHSMKEGIHA |
| | | QQKEPMIGVNQELAYFYPELFRQFYQLDAYPSGAW |
| | | YYVPLGTQYTDAPSFSDIPNPIGSENSEKTTMPLW |
| 2 | Bovine Alpha-S1 Casein | RPKHPIKHQGLPQEVLNENLLRFFVAPFPEVFGKEK |
| | Mature protein | VNELSKDIGSESTEDQAMEDIKQMEAESISSSEEIVP |
| | | NSVEQKHIQKEDVPSERYLGYLEQLLRLKKYKVPQ |
| | | LEIVPNSAEERLHSMKEGIHAQQKEPMIGVNQELAY |
| | | FYPELFRQFYQLDAYPSGAWYYVPLGTQYTDAPSF |
| | | SDIPNPIGSENSEKTTMPLW |
| 3 | Ovine Alpha S1 casein | MKLLILTCLVAVALARPKHPIKHQGLSSEVLNENL |
| | | LRFVVAPFPEVFRKENINELSKDIGSESIEDQAMEDA |
| | | KQMKAGSSSSSEEIVPNSAEQKYIQKEDVPSERYLG |
| | | YLEQLLRLKKYNVPQLEIVPKSAEEQLHSMKEGNP |
| | | AHQKQPMIAVNQELAYFYPQLFRQFYQLDAYPSGA |
| | | WYYLPLGTQYTDAPSFSDIPNPIGSENSGKITMPLW |
| 4 | Ovine Alpha S1 casein | RPKHPIKHQGLSSEVLNENLLRFVVAPFPEVFRKENI |
| | Mature protein | NELSKDIGSESIEDQAMEDAKQMKAGSSSSSEEIVP |
| | | NSAEQKYIQKEDVPSERYLGYLEQLLRLKKYNVPQ |
| | | LEIVPKSAEEQLHSMKEGNPAHQKQPMIAVNQELA |
| | | YFYPQLFRQFYQLDAYPSGAWYYLPLGTQYTDAPS |
| | | FSDIPNPIGSENSGKITMPLW |
| 5 | Caprine Alpha S1 casein | MKLLILTCLVAVALARPKHPINHRGLSPEVPNENL |
| | | LRFVVAPFPEVFRKENINELSKDIGSESTEDQAMED |
| | | AKQMKAGSSSSSEEIVPNSAEQKYIQKEDVPSERYL |
| | | GYLEQLLRLKKYNVPQLEIVPKSAEEQLHSMKEGN |
| | | PAHQKQPMIAVNQELAYFYPQLFRQFYQLDAYPSG |
| | | AWYYLPLGTQYTDAPSFSDIPNPIGSENSGKTTMPL |
| | | W |
| 6 | Caprine Alpha S1 casein | RPKHPINHRGLSPEVPNENLLRFVVAPFPEVFRKENI |
| | Mature Protein | NELSKDIGSESTEDQAMEDAKQMKAGSSSSSEEIVP |
| | | NSAEQKYIQKEDVPSERYLGYLEQLLRLKKYNVPQ |
| | | LEIVPKSAEEQLHSMKEGNPAHQKQPMIAVNQELA |
| | | YFYPQLFRQFYQLDAYPSGAWYYLPLGTQYTDAPS |
| | | FSDIPNPIGSENSGKTTMPLW |
| 7 | Buffalo Alpha S1 | MKLLILTCLVAVALARPKQPIKHQGLPQGVLNEN |
| | Casein | LLRFFVAPFPEVFGKEKVNELSTDIGSESTEDQAME |
| | | DIKQMEAESISSSEEIVPISVEQKHIQKEDVPSERYLG |
| | | YLEQLLRLKKYNVPQLEIVPNLAEEQLHSMKEGIH |
| | | AQQKEPMIGVNQELAYFYPQLFRQFYQLDAYPSGA |
| | | WYYVPLGTQYPDAPSFSDIPNPIGSENSEKTTMPLW |
| 8 | Buffalo Alpha S1 | RPKQPIKHQGLPQGVLNENLLRFFVAPFPEVFGKEK |
| | Casein Mature Protein | VNELSTDIGSESTEDQAMEDIKQMEAESISSSEEIVPI |
| | | SVEQKHIQKEDVPSERYLGYLEQLLRLKKYNVPQL |
| | | EIVPNLAEEQLHSMKEGIHAQQKEPMIGVNQELAY |
| | | FYPQLFRQFYQLDAYPSGAWYYVPLGTQYPDAPSF |
| | | SDIPNPIGSENSEKTTMPLW |
| 9 | Equine Alpha S1 Casein | MKLLILTCLVAVALARPKLPHRQPEIIQNEQDSRE |
| | | KVLKERKFPSFALEYINELNRQRELLKEKQKDEHKE |
| | | YLIEDPEQQESSSTSSSEEVVPINTEQKRIPREDMLY |
| | | QHTLEQLRRLSKYNQLQLQAIHAQEQLIRMKENSQ |
| | | RKPMRVVNQEQAYFYLEPFQPSYQLDVYPYAAWF |
| | | HPAQIMQHVAYSPFHDTAKLIASENSEKTDIIPEW |
| 10 | Equine Alpha S1 Casein | RPKLPHRQPEIIQNEQDSREKVLKERKFPSFALEYIN |
| | Mature Protein | ELNRQRELLKEKQKDEHKEYLIEDPEQQESSSTSSS |
| | | EEVVPINTEQKRIPREDMLYQHTLEQLRRLSKYNQL |
| | | QLQAIHAQEQLIRMKENSQRKPMRVVNQEQAYFY |
| | | LEPFQPSYQLDVYPYAAWFHPAQIMQHVAYSPFHD |
| | | TAKLIASENSEKTDIIPEW |
| 11 | Camel Alpha S1 casein | MKLLILTCLVAVALARPKYPLRYPEVFQNEPDSIE |
| | | EVLNKRKILELAVVSPIQFRQENIDELKDTRNEPTED |
| | | HIMEDTERKESGSSSSEEVVSSTTEQKDILKEDMPS |
| | | QRYLEELHRLNKYKLLQLEAIRDQKLIPRVKLSSHP |
| | | YLEQLYRINEDNHPQLGEPVKVVTQEQAYFHLEPFP |
| | | QFFQLGASPYVAWYYPPQVMQYIAHPSSYDTPEGI |
| | | ASEDGGKTDVMPQWW |
| 12 | Camel Alpha S1 casein | RPKYPLRYPEVFQNEPDSIEEVLNKRKILELAVVSPI |
| | Mature Protein | QFRQENIDELKDTRNEPTEDHIMEDTERKESGSSSSE |
| | | EVVSSTTEQKDILKEDMPSQRYLEELHRLNKYKLL |
| | | QLEAIRDQKLIPRVKLSSHPYLEQLYRINEDNHPQL |
| | | GEPVKVVTQEQAYFHLEPFPQFFQLGASPYVAWYY |
| | | PPQVMQYIAHPSSYDTPEGIASEDGGKTDVMPQW |
| | | W |
| 13 | Human Alpha S1 Casein | MRLLILTCLVAVALARPKLPLRYPERLQNPSESSEP |
| | | IPLESREEYMNGMNRQRNILREKQTDEIKDTRNEST |
| | | QNCVVAEPEKMESSISSSSEEMSLSKCAEQFCRLNE |
| | | YNQLQLQAAHAQEQIRRMNENSHVQVPFQQLNQL |
| | | AAYPYAVWYYPQIMQYVPFPPFSDISNPTAHENYE |
| | | KNNVMLQW |
| 14 | Human Alpha S1 Casein | RPKLPLRYPERLQNPSESSEPIPLESREEYMNGMNR |
| | Mature Protein | QRNILREKQTDEIKDTRNESTQNCVVAEPEKMESSI |
| | | SSSSEEMSLSKCAEQFCRLNEYNQLQLQAAHAQEQI |
| | | RRMNENSHVQVPFQQLNQLAAYPYAVWYYPQIMQ |
| | | YVPFPPFSDISNPTAHENYEKNNVMLQW |
| 15 | Bovine Alpha-S2 Casein | MKFFIFTCLLAVALAKNTMEHVSSSEESIISQETYK |
| | | QEKNMAINPSKENLCSTFCKEVVRNANEEEYSIGSS |
| | | SEESAEVATEEVKITVDDKHYQKALNEINQFYQKFP |
| | | QYLQYLYQGPIVLNPWDQVKRNAVPITPTLNREQL |
| | | STSEENSKKTVDMESTEVFTKKTKLTEEEKNRLNFL |
| | | KKISQRYQKFALPQYLKTVYQHQKAMKPWIQPKT |
| | | KVIPYVRYL |
| 16 | Bovine Alpha-S2 Casein | KNTMEHVSSSEESIISQETYKQEKNMAINPSKENLC |
| | Mature protein | STFCKEVVRNANEEEYSIGSSSEESAEVATEEVKITV |
| | | DDKHYQKALNEINQFYQKFPQYLQYLYQGPIVLNP |
| | | WDQVKRNAVPITPTLNREQLSTSEENSKKTVDMES |
| | | TEVFTKKTKLTEEEKNRLNFLKKISQRYQKFALPQY |
| | | LKTVYQHQKAMKPWIQPKTKVIPYVRYL |
| 17 | Ovine Alpha S2 casein | MKFFIFTCLLAVALAKHKMEHVSSSEEPINISQEIY |
| | | KQEKNMAIHPRKEKLCTTSCEEVVRNADEEEYSIRS |
| | | SSEESAEVAPEEVKITVDDKHYQKALNEINQFYQKF |
| | | PQYLQYLYQGPIVLNPWDQVKRNAGPFTPTVNREQ |
| | | LSTSEENSKKTIDMESTEVFTKKTKLTEEEKNRLNF |
| | | LKKISQYYQKFAWPQYLKTVDQHQKAMKPWTQP |
| | | KTNAIPYVRYL |
| 18 | Ovine Alpha S2 casein | KHKMEHVSSSEEPINISQEIYKQEKNMAIHPRKEKL |
| | Mature protein | CTTSCEEVVRNADEEEYSIRSSSEESAEVAPEEVKIT |
| | | VDDKHYQKALNEINQFYQKFPQYLQYLYQGPIVLN |
| | | PWDQVKRNAGPFTPTVNREQLSTSEENSKKTIDME |
| | | STEVFTKKTKLTEEEKNRLNFLKKISQYYQKFAWP |
| | | QYLKTVDQHQKAMKPWTQPKTNAIPYVRYL |
| 19 | Caprine Alpha S2 casein | MKFFIFTCLLAVALAKHKMEHVSSSEEPINIFQEIY |
| | | KQEKNMAIHPRKEKLCTTSCEEVVRNANEEEYSIRS |
| | | SSEESAEVAPEEIKITVDDKHYQKALNEINQFYQKF |
| | | PQYLQYPYQGPIVLNPWDQVKRNAGPFTPTVNREQ |
| | | LSTSEENSKKTIDMESTEVFTKKTKLTEEEKNRLNF |
| | | LKKISQYYQKFAWPQYLKTVDQHQKAMKPWTQP |
| | | KTNAIPYVRYL |
| 20 | Caprine Alpha S2 casein | KHKMEHVSSSEEPINIFQEIYKQEKNMAIHPRKEKL |
| | Mature Protein | CTTSCEEVVRNANEEEYSIRSSSEESAEVAPEEIKITV |
| | | DDKHYQKALNEINQFYQKFPQYLQYPYQGPIVLNP |
| | | WDQVKRNAGPFTPTVNREQLSTSEENSKKTIDMES |
| | | TEVFTKKTKLTEEEKNRLNFLKKISQYYQKFAWPQ |
| | | YLKTVDQHQKAMKPWTQPKTNAIPYVRYL |
| 21 | Buffalo Alpha S2 | MKFFIFTCLLAVALAKHTMEHVSSSEESIISQETYK |
| | Casein | QEKNMAIHPSKENLCSTFCKEVIRNANEEEYSIGSSS |
| | | EESAEVATEEVKITVDDKHYQKALNEINQFYQKFP |
| | | QYLQYLYQGPIVLNPWDQVKRNAVPITPTLNREQL |
| | | STSEENSKKTVDMESTEVFTKKTKLTEEDKNRLNFL |
| | | KKISQHYQKFAWPQYLKTVYQYQKAMKPWTQPK |
| | | TNVIPYVRYL |
| 22 | Buffalo Alpha S2 | KHTMEHVSSSEESIISQETYKQEKNMAIHPSKENLC |
| | Casein Mature Protein | STFCKEVIRNANEEEYSIGSSSEESAEVATEEVKITV |
| | | DDKHYQKALNEINQFYQKFPQYLQYLYQGPIVLNP |
| | | WDQVKRNAVPITPTLNREQLSTSEENSKKTVDMES |
| | | TEVFTKKTKLTEEDKNRLNFLKKISQHYQKFAWPQ |
| | | YLKTVYQYQKAMKPWTQPKTNVIPYVRYL |
| 23 | Equine Alpha S2 Casein | MKFFIFTCLLAVALAKHNMEHRSSSEDSVNISQEK |
| | | FKQEKYVVIPTSKESICSTSCEEATRNINEMESAKFP |
| | | TEREEKEVEEKHHLKQLNKINQFYEKLNFLQYLQA |
| | | LRQPRIVLTPWDQTKTGDSPFIPIVNTEQLFTSEEIPK |
| | | KTVDMESTEVVTEKTELTEEEKNYLKLLYYEKFTL |
| | | PQYFKIVRQHQTTMDPRSHRKTNSYQIIPVLRYF |
| 24 | Equine Alpha S2 Casein | KHNMEHRSSSEDSVNISQEKFKQEKYVVIPTSKESIC |
| | Mature Protein | STSCEEATRNINEMESAKFPTEREEKEVEEKHHLKQ |
| | | LNKINQFYEKLNFLQYLQALRQPRIVLTPWDQTKT |
| | | GDSPFIPIVNTEQLFTSEEIPKKTVDMESTEVVTEKT |
| | | ELTEEEKNYLKLLYYEKFTLPQYFKIVRQHQTTMD |
| | | PRSHRKTNSYQIIPVLRYF |
| 25 | Camel Alpha S2 casein | MKFFIFTCLLAVVLAKHEMDQGSSSEESINVSQQK |
| | | FKQVKKVAIHPSKEDICSTFCEEAVRNIKEVESAEVP |
| | | TENKISQFYQKWKFLQYLQALHQGQIVMNPWDQG |
| | | KTRAYPFIPTVNTEQLSISEESTEVPTEESTEVFTKKT |
| | | ELTEEEKDHQKFLNKIYQYYQTFLWPEYLKTVYQY |
| | | QKTMTPWNHIKRYF |
| 26 | Camel Alpha S2 casein | KHEMDQGSSSEESINVSQQKFKQVKKVAIHPSKEDI |
| | Mature Protein | CSTFCEEAVRNIKEVESAEVPTENKISQFYQKWKFL |
| | | QYLQALHQGQIVMNPWDQGKTRAYPFIPTVNTEQL |
| | | SISEESTEVPTEESTEVFTKKTELTEEEKDHQKFLNK |
| | | IYQYYQTFLWPEYLKTVYQYQKTMTPWNHIKRYF |
| 27 | Bovine Kappa Casein | MMKSFFLVVTILALTLPFLGAQEQNQEQPIRCEK |
| | | DERFFSDKIAKYIPIQYVLSRYPSYGLNYYQQKPVA |
| | | LINNQFLPYPYYAKPAAVRSPAQILQWQVLSNTVP |
| | | AKSCQAQPTTMARHPHPHLSFMAIPPKKNQDKTEIP |
| | | TINTIASGEPTSTPTTEAVESTVATLEDSPEVIESPPEI |
| | | NTVQVTSTAV |
| 28 | Bovine Kappa Casein | QEQNQEQPIRCEKDERFFSDKIAKYIPIQYVLSRYPS |
| | Mature protein | YGLNYYQQKPVALINNQFLPYPYYAKPAAVRSPAQ |
| | | ILQWQVLSNTVPAKSCQAQPTTMARHPHPHLSFMA |
| | | IPPKKNQDKTEIPTINTIASGEPTSTPTTEAVESTVAT |
| | | LEDSPEVIESPPEINTVQVTSTAV |
| 29 | Ovine Kappa casein | MMKSFFLVVTILALTLPFLGAQEQNQEQRICCEK |
| | | DERFFDDKIAKYIPIQYVLSRYPSYGLNYYQQRPVA |
| | | LINNQFLPYPYYAKPVAVRSPAQTLQWQVLPNAVP |
| | | AKSCQDQPTAMARHPHPHLSFMAIPPKKDQDKTEI |
| | | PAINTIASAEPTVHSTPTTEAVVNAVDNPEASSESIA |
| | | SAPETNTAQVTSTEV |
| 30 | Ovine Kappa casein | QEQNQEQRICCEKDERFFDDKIAKYIPIQYVLSRYPS |
| | Mature protein | YGLNYYQQRPVALINNQFLPYPYYAKPVAVRSPAQ |
| | | TLQWQVLPNAVPAKSCQDQPTAMARHPHPHLSFM |
| | | AIPPKKDQDKTEIPAINTIASAEPTVHSTPTTEAVVN |
| | | AVDNPEASSESIASAPETNTAQVTSTEV |
| 31 | Caprine Kappa casein | MMKSFFLVVTILALTLPFLGAQEQNQEQPICCEK |
| | | DERFFDDKIAKYIPIQYVLSRYPSYGLNYYQQRPVA |
| | | LINNQFLPYPYYAKPVAVRSPAQTLQWQVLPNTVP |
| | | AKSCQDQPTTLARHPHPHLSFMAIPPKKDQDKTEV |
| | | PAINTIASAEPTVHSTPTTEAIVNTVDNPEASSESIAS |
| | | ASETNTAQVTSTEV |
| 32 | Caprine Kappa casein | QEQNQEQPICCEKDERFFDDKIAKYIPIQYVLSRYPS |
| | Mature Protein | YGLNYYQQRPVALINNQFLPYPYYAKPVAVRSPAQ |
| | | TLQWQVLPNTVPAKSCQDQPTTLARHPHPHLSFMA |
| | | IPPKKDQDKTEVPAINTIASAEPTVHSTPTTEAIVNT |
| | | VDNPEASSESIASASETNTAQVTSTEV |
| 33 | Buffalo Kappa Casein | MMKSFFLVVTILALTLPFLGAQEQNQEQPIRCEKE |
| | | ERFFNDKIAKYIPIQYVLSRYPSYGLNYYQQKPVALI |
| | | NNQFLPYPYYAKPAAVRSPAQILQWQVLPNTVPAK |
| | | SCQAQPTTMTRHPHPHLSFMAIPPKKNQDKTEIPTI |
| | | NTIVSVEPTSTPTTEAIENTVATLEASSEVIESVPETN |
| | | TAQVTSTVV |
| 34 | Buffalo Kappa Casein | QEQNQEQPIRCEKEERFFNDKIAKYIPIQYVLSRYPS |
| | Mature Protein | YGLNYYQQKPVALINNQFLPYPYYAKPAAVRSPAQ |
| | | ILQWQVLPNTVPAKSCQAQPTTMTRHPHPHLSFMA |
| | | IPPKKNQDKTEIPTINTIVSVEPTSTPTTEAIENTVAT |
| | | LEASSEVIESVPETNTAQVTSTVV |
| 35 | Equine Kappa Casein | MKSFFLVVNILALTLPFLGAEVQNQEQPTCHKND |
| | | ERFFDLKTVKYIPIYYVLNSSPRYEPIYYQHRLALLI |
| | | NNQHMPYQYYARPAAVRPHVQIPQWQVLPNIYPST |
| | | VVRHPCPHPSFIAIPPKKLQEITVIPKINTIATVEPTPIP |
| | | TPEPTVNNAVIPDASSEFIIASTPETTTVPVTSPVVQK |
| | | L |
| 36 | Equine Kappa Casein | EVQNQEQPTCHKNDERFFDLKTVKYIPIYYVLNSSP |
| | Mature Protein | RYEPIYYQHRLALLINNQHMPYQYYARPAAVRPHV |
| | | QIPQWQVLPNIYPSTVVRHPCPHPSFIAIPPKKLQEIT |
| | | VIPKINTIATVEPTPIPTPEPTVNNAVIPDASSEFIIAST |
| | | PETTTVPVTSPVVQKL |
| 37 | Camel Kappa casein | MKSFFLVVTILALTLPFLGAEVQNQEQPTCFEKVE |
| | | RLLNEKTVKYFPIQFVQSRYPSYGINYYQHRLAVPI |
| | | NNQFIPYPNYAKPVAIRLHAQIPQCQALPNIDPPTVE |
| | | RRPRPRPSFIAIPPKKTQDKTVNPAINTVATVEPPVIP |
| | | TAEPAVNTVVIAEASSEFITTSTPETTTVQITSTEI |
| 38 | Camel Kappa casein | EVQNQEQPTCFEKVERLLNEKTVKYFPIQFVQSRYP |
| | Mature Protein | SYGINYYQHRLAVPINNQFIPYPNYAKPVAIRLHAQI |
| | | PQCQALPNIDPPTVERRPRPRPSFIAIPPKKTQDKTV |
| | | NPAINTVATVEPPVIPTAEPAVNTVVIAEASSEFITTS |
| | | TPETTTVQITSTEI |
| 39 | Human Kappa Casein | MKSFLLVVNALALTLPFLAVEVQNQKQPACHEN |
| | | DERPFYQKTAPYVPMYYVPNSYPYYGTNLYQRRP |
| | | AIAINNPYVPRTYYANPAVVRPHAQIPQRQYLPNSH |
| | | PPTVVRRPNLHPSFIAIPPKKIQDKIIIPTINTIATVEPT |
| | | PAPATEPTVDSVVTPEAFSESIITSTPETTTVAVTPPT |
| | | A |
| 40 | Human Kappa Casein | EVQNQKQPACHENDERPFYQKTAPYVPMYYVPNS |
| | Mature Protein | YPYYGTNLYQRRPAIAINNPYVPRTYYANPAVVRP |
| | | HAQIPQRQYLPNSHPPTVVRRPNLHPSFIAIPPKKIQ |
| | | DKIIIPTINTIATVEPTPAPATEPTVDSVVTPEAFSESII |
| | | TSTPETTTVAVTPPTA |
| 41 | Bovine Beta Casein | MKVLILACLVALALARELEELNVPGEIVESLSSSEE |
| | | SITRINKKIEKFQSEEQQQTEDELQDKIHPFAQTQSL |
| | | VYPFPGPIPNSLPQNIPPLTQTPVVVPPFLQPEVMGV |
| | | SKVKEAMAPKHKEMPFPKYPVEPFTESQSLTLTDV |
| | | ENLHLPLPLLQSWMHQPHQPLPPTVMFPPQSVLSLS |
| | | QSKVLPVPQKAVPYPQRDMPIQAFLLYQEPVLGPV |
| | | RGPFPIIV |
| 42 | Bovine Beta Casein | RELEELNVPGEIVESLSSSEESITRINKKIEKFQSEEQ |
| | Mature Protein | QQTEDELQDKIHPFAQTQSLVYPFPGPIPNSLPQNIP |
| | | PLTQTPVVVPPFLQPEVMGVSKVKEAMAPKHKEM |
| | | PFPKYPVEPFTESQSLTLTDVENLHLPLPLLQSWMH |
| | | QPHQPLPPTVMFPPQSVLSLSQSKVLPVPQKAVPYP |
| | | QRDMPIQAFLLYQEPVLGPVRGPFPIIV |

### Embodiments

### Examples

The following illustrative examples are representative of embodiments of the compositions and methods described herein and are not meant to be limiting in any way.

### Example 1: Expression of Casein Proteins in Lactococcus lactis Via Nisin-Inducible System (NICE)

### Constructs Design, Cloning and Transformation

Bovine kappa casein (variant B) and bovine alpha-S1-casein (variant C) protein coding sequences (without the native signal peptide) were codon-optimized for expression in Lactococcus lactis and a synthetic operon was constructed for co-expression and secretion of the two proteins under a nisin-inducible promoter. Signal peptide sequence from natively secreting lactococcal protein Usp45 was used to drive protein secretionA synthetic operon was then cloned into an E. coli custom vector via restriction digest compatible sites and confirmed via Sanger sequencing, from which it was subcloned into nisin-inducible pNZ8149 vector via restriction digestion and ligation. The vector was transformed into compatible L. lactis strain NZ3900 via electroporation and completely defined media (CDM) supplemented with lactose was used for selection. Positive clones were confirmed via colony PCR and 3 positive clones were taken forward for the protein expression induction and analysis.

### Protein Expression and Analysis

Individual colonies were grown at 30° C. in liquid culture and protein production was induced with nisin for 2.5 hours (control samples left uninduced). Cells were then harvested by centrifugation and TCA-precipitated supernatants and lysed cell pellets were analysed by Coomassie gel staining (SDS-PAGE) and chemiluminescence (Western Blot against kappa-casein and alpha-S1-casein, LSBio primary antibodies). Kappa-casein expression in L. lactis was detected in the tested transformants by Coomassie stained protein gel and western blot.

### Example 2: Expression in L. lactis Via pH-Inducible System

Similar to the constructions above, casein protein constructions were created for alpha, beta and kappa casein replacing the nisin promoter with the P170 promoter, a pH/lactate inducible promoter for L. lactis. Each of these constructs contained a secretion signal peptide.

Both alpha-S1 and kappa casein were detected in L. lactis upon secretion on western blot. Protein product accumulated intracellularly for alpha-S1-casein. Alpha-S1-casein secreted poorly, whereas kappa casein showed near-complete secretion of protein produced.

### Example 3: Expression in B. subtilis

### Constructs Design, Cloning and Transformation

Bovine alpha-S1-casein (variant C) protein coding sequence (without the native signal peptide) His-tagged C-terminally was codon-optimized for expression in Bacillus subtilis. Constructs were created with and without the codon-optimized signal peptide of amyQ, alpha-amylase Bacillus amyloliquefaciens which has been reported for the efficient secretion of recombinant proteins. Constructs were cloned through E. coli via Gibson cloning into transformation and expression IPTG-inducible vector pHT01 and confirmed via Sanger sequencing. pHT01 is an E. coli/B. subtilis shuttle vector that provides ampicillin resistance to E. coli and chloramphenicol resistance to B. subtilis. Positive clones were further transformed into chemically competent B. subtilis WB800N. Positive clones were confirmed via colony PCR and 3 positive clones were taken forward for the protein expression induction and analysis.

### Protein Expression and Analysis

Individual colonies were grown at 37° C. in liquid culture and protein production was induced with IPTG for 1 hour, 2 hours and 6 hours (control samples were left uninduced). Cells were then harvested by centrifugation, and TCA-precipitated supernatants and lysed cell pellets were analysed by Coomassie gel staining (SDS-PAGE) and chemiluminescence (Western Blot against His tag and alpha-S1-casein).

Western blotting showed expression of the alpha-S1-casein in B. subtilis.

### Example 4: Expression in E. coli

### Constructs Design, Cloning and Transformation

Bovine alpha-S1-casein (variant C) protein coding sequence (without the native signal peptide) codon-optimized for Escherichia coli was cloned into IPTG-inducible commercially available pET vectors. Cloning was performed via Gibson reaction of DNA fragments and vector in such a way that only the protein coding sequence was left within the open reading frame. Gibson reactions were transformed into competent cells and confirmed by Sanger sequencing. Vectors were then transformed into chemically competent E. coli BL21(DE3) cells, or their derivatives (e.g. BL21-pLysS), and several single colonies were screened for expression.

### Protein Expression, Analysis and Purification

Individual colonies were grown at 37 C in liquid culture, and protein production was induced with IPTG for 4 hours. Cells were then harvested by centrifugation, and lysed cell pellets were analysed by Coomassie gel staining (SDS-PAGE) and chemiluminescence (Western Blot against alpha-S1-casein). For protein purification, the insoluble fraction was removed by centrifugation and the soluble fraction was then precipitated with ammonium sulfate at room temperature and pelleted by centrifugation. The pellet was resuspended in urea, followed by dialysis against disodium phosphate. The insoluble proteins were removed by centrifugation, and the remaining contaminants were removed by precipitation with ethanol and ammonium acetate followed by centrifugation. The resulting alpha-S1-casein solution was concentrated using a centrifugal filtration unit and then dialyzed against disodium phosphate. Purified product was analysed on a Coomassie stained gel similarly to explained above.

Alpha-S1-casein was expressed intracellularly in E. coli, successfully detected on Coomassie stained protein gel and purified.

### Example 5: Cheesemaking Processes Using Micellar Casein

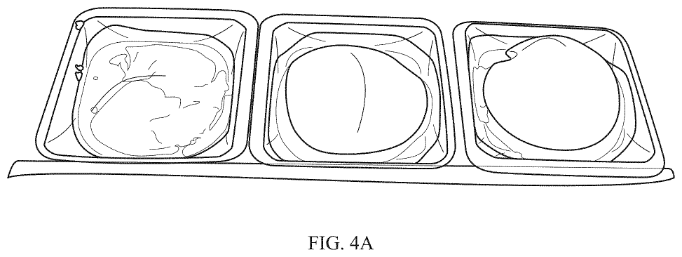

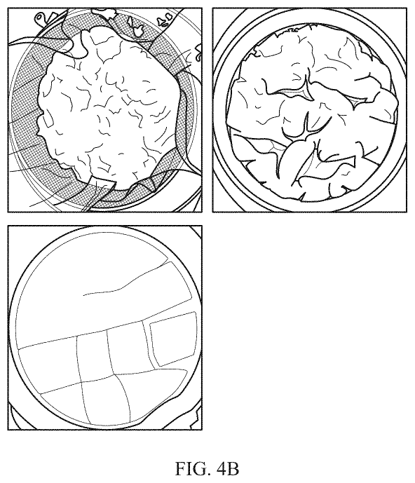

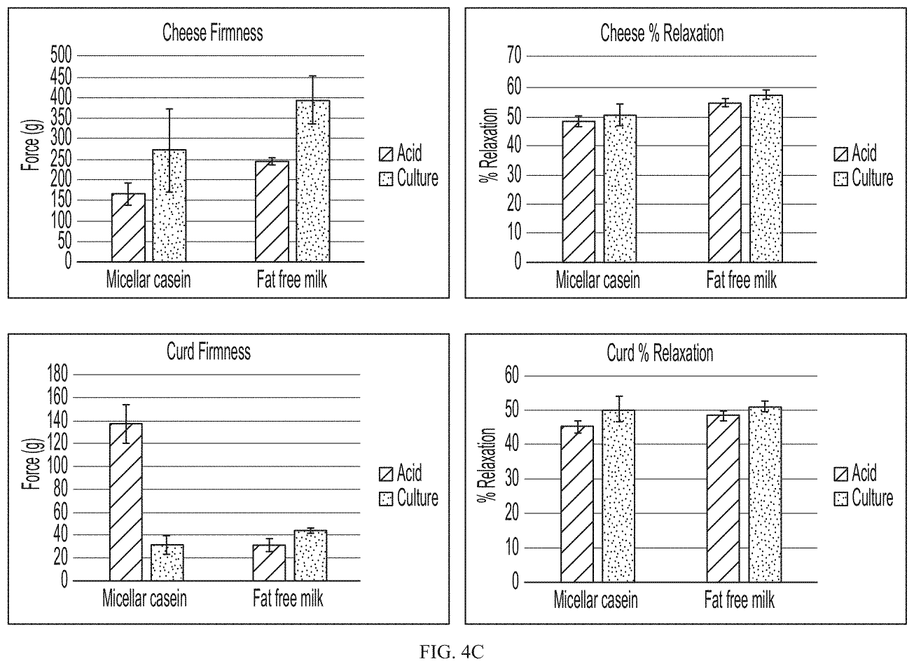

In this example, pasta filata cheese-like material was made from micellar casein powder. Micellar casein is typically obtained in industry by ultrafiltration of skim milk to isolate casein micelles and spray drying techniques to powderize casein micelles. Micellar casein that was mixed with water and sugar acted similar to milk in the cheesemaking process with bacteria fermentation or acid addition, and rennet (chymosin), and it resulted with milk-like cheese, which was specifically turned into a mozzarella-like cheese (FIG. 4A).

In similar way, using micellar casein (purchased from Milk Specialties Global), a mozzarella-like material was made through numerous methods: 14 g-28 g micellar casein powder, 1000 ml water, mozzarella-like cheese with rennet and citric acid; 14 g-28 g micellar casein powder, 1000 ml water, mozzarella-like cheese with just citric acid; 14 g-28 g micellar casein powder, 1000 ml water, 20-55 g plant-based sugar, mozzarella-like cheese with rennet and lactic acid bacteria; 14 g-28 g micellar casein powder, 1-4% plant-based fat in a stable emulsion (with and without emulsifier), 20-55 g plant-based sugar, mozzarella-like cheese with rennet and citric acid; 14 g-28 g micellar casein powder, 1-4% plant-based fat in a stable emulsion (with and without emulsifier), 20-55 g plant-based sugar, mozzarella-like cheese with only citric acid; 14 g-28 g micellar casein powder, 1-4% plant-based fat in a stable emulsion (with and without emulsifier), 20-55 g plant-based sugar, mozzarella-like cheese with rennet and lactic acid bacteria.

In another example, micellar casein liquid colloid (2.8%) supplemented with lactose (5%) was acidified using mesophilic bacterial starter culture, in parallel with fat-free milk as a control. Micellar casein colloid and milk were acidified down to pH ˜5.7, when renneting agent was added and acidified colloids were left undisturbed until the curd settled (FIG. 4B). Curds were then drained through a cheese cloth, dipped in hot water and stretched into mozzarella-like cheese balls. Texture analyzer assessment of firmness of samples is shown in FIG. 4C (mean with standard deviation on three replicate samples).

### Example 6: Formulation and Properties of Mozzarella-Like Cheese Made Using Micellar Casein

In this example, micellar casein (3.3% final w/v), soy lecithin (0.1% final w/v), melted coconut oil (1% final w/v) and melted margarine (1% final w/v) were blended together into a paste. The paste was mixed with 40° C. Milli-Q water and stirred to incorporate, after which maltose (2.5% final w/v) was mixed in. Blend was mixed with a high sheer mixer until fat was incorporated. Liquid was cooled to 33° C. and citric acid solution (0.15% final w/v) was added with vigorous stirring, after which rennet solution (0.0036% final v/v) was mixed in and the mixture was allowed to stand for 15-30 mins. Curd was drained in a cheesecloth-lined sieve and immersed into hot water (>60° C.), stretched and folded a few times, after which it was shaped into a mozzarella-like ball.

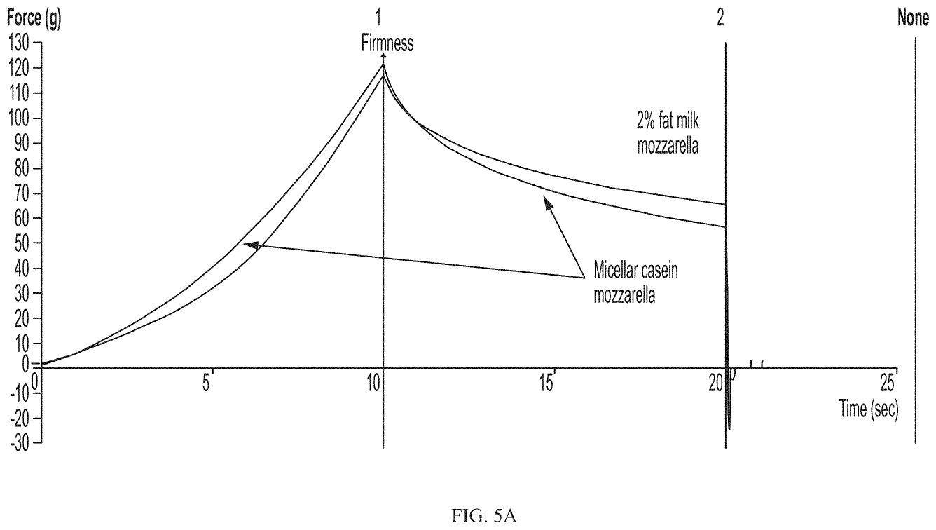

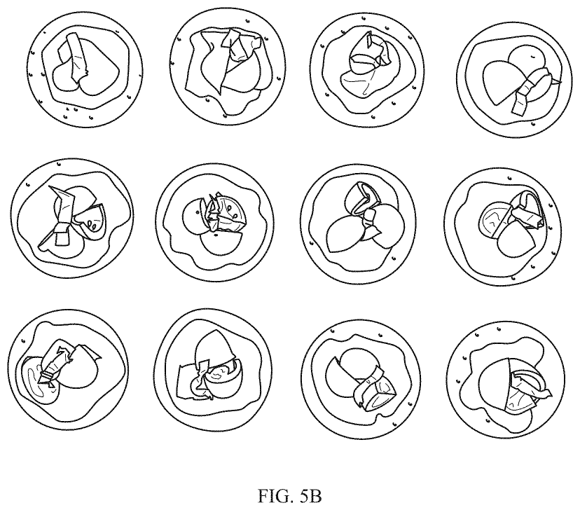

The texture profile of made mozzarella-like cheese was probed on Texture Analyzer TX.TA using a stress-relaxation test and compared with mozzarella made from 2% fat store-bought milk. FIG. 5A shows that the texture profile of mozzarella-like cheese made from micellar casein in the formulation above matches very closely to the texture of mozzarella made from 2% milk.

Mozzarella-like cheese made from micellar casein was evaluated melted on a ‘pizza’ (homemade crust with no sauce and a small amount of cherry tomatoes, basil and olive oil) in a triangle test against store-bought fresh mozzarella as shown in FIG. 5B. In a triangle test, tasters are given three samples, where two are the same and one is different. They are asked to taste all three and identify the odd sample out. The odds of guessing correctly are 1 in 3 (33.33%), so if the rate of correct responses is significantly greater, one can conclude that there is a discernible difference between the two samples. If the rate of correct responses is not significantly greater than 33.33%, one can conclude that there is no discernible difference between the two samples.

Samples were presented to tasters in a random order, with half the tasters receiving two of the micellar casein mozzarella pizzas and one store-bought mozzarella pizza, and the other half receiving the reverse. Samples were identified by a three-digit code only.

Of 19 tasters, 6 correctly identified the odd one out, meaning the rate of correct responses was 31.6%. This suggests no significant difference was identified between the micellar casein mozzarella-like cheese and store-bought mozzarella cheese when melted on pizza.

### Example 6: Casein Micelles/Liquid Colloid Reconstitution Using Alpha, Beta and Kappa Casein

Alpha-casein, beta-casein and kappa-casein fractions were purchased as lyophilized powders from Sigma-Aldrich.

The amounts of protein used in the micelle/liquid colloid forming experiments were 1.4% (0.5× milk concentration for casein), 2.8% (1× milk concentration for casein), or 3.2% (1× milk concentration for total protein, w/v). Unless otherwise noted, for experiments with all 3 caseins, 15% of the total protein (by mass) was kappa-casein, 30% of the total protein was beta-casein and 55% of the total protein was alpha-casein. This gives the following amounts for each condition shown in Table 2.

TABLE 2
Protein concentrations

| % Protein | alpha casein | beta casein | kappa casein |
|---|---|---|---|
| (total) (w/v) | (mg/ml) | (mg/ml) | (mg/ml) |
| 1.4 | 7.7 | 4.2 | 2.1 |
| 2.8 | 15.4 | 8.4 | 4.2 |
| 3.2 | 17.6 | 9.6 | 4.8 |

Alpha-casein, beta-casein and kappa casein were added sequentially to water and stirred until fully dissolved. In some experiments, the mixture was also incubated at room temperature overnight.

Micelle Induction with Salt Addition

Alpha, beta and kappa casein were subject to a series of salt combinations to induce micelles, where the ratio of calcium, phosphate and citrate was kept at 3:2:1 or 6:4:1 and where the calcium concentration is 14-24 mM for 1.4% total casein. The resulting solutions were evaluated using DLS, absorbance and cheesemaking.

TABLE 3
Final salt concentrations (mM) for 1.4% total protein concentration

| | A | B | C | D | E | F |
|---|---|---|---|---|---|---|
| CaCl2 | 16.8 | 16.8 | 16.8 | 18.5 | 18.5 | 16.8 |
| K2HPO4 | 11.2 | 11.2 | 11.2 | 12.4 | 12.4 | 11.2 |
| K3 citrate | 5.6 | 2.8 | 4.2 | 6.2 | 5.6 | 0 |

Filtered (220 nm) calcium (CaCl2 if not stated otherwise), phosphate (K2HPO4 if not stated otherwise), and citrate (K3 Citrate if not stated otherwise) were titrated into casein solution in five additions, over a fixed addition schedule, to final concentrations from Table 3. First addition comprised only a fraction of calcium, and other additions equal fractions of all three salts.

### Particle Size Measurement Using Dynamic Light Scattering (DLS) Instrument

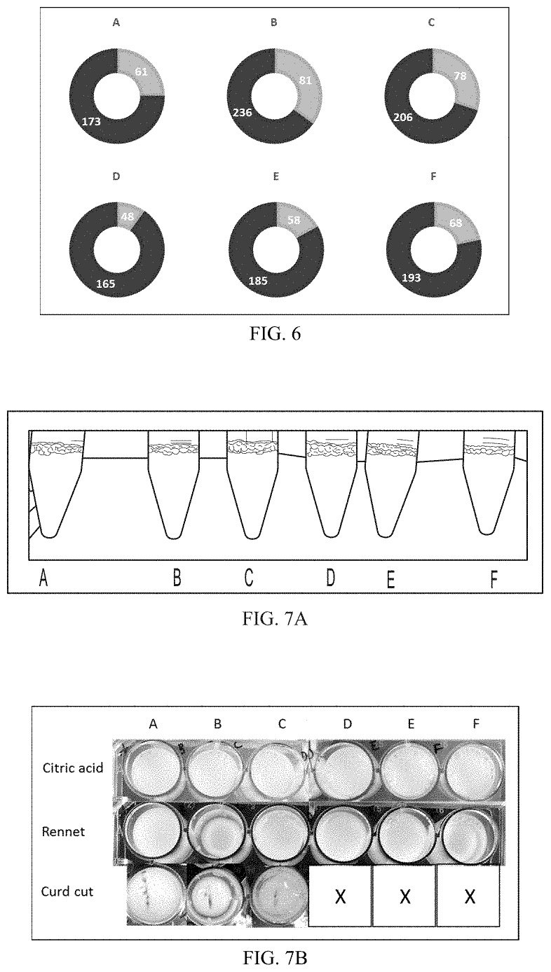

To provide better accuracy of measurement, samples were diluted generally to a concentration of 0.14% (or 1.4 mg/mL) or less in filtered (220 nm) milliQ water. Samples were measured using either Entegris Nicomp or Malvern Zetaseizer instrument. On Nicomp, three replicates were measured at a 90° detection angle and data was analyzed using the Nicomp analysis software. On Zetasizer, three replicates were measured at a 173° detection angle and data was analyzed using the Zetasizer's small peak analysis mode. Doughnut charts (FIG. 6) show the particle sizing data for an experiment where micelles were reconstituted from individual casein proteins at 1.4% protein concentration. Gaussian-like peaks are reported by the instrument for any major light scattering particle population. Mean of a peak gives the particle (micelle) diameter in nanometers (nm), and intensity of a peak gives relative scattering compared to other peaks reported. The doughnut charts also report the peak mean—particle size in nm—as numbers on slices of doughnut charts, and their intensities—relative amounts as the proportion of slice as a part of the doughnut chart (angle). Doughnut charts show average particle size and average intensity across three replicate measurements. In cow milk, casein micelle is the predominant particle detected with a size typically from 150 to 300 nm, and a maximum generally up to 500 nm, accompanied by sub-micellar particles detected with sizes from 30 to 80 nm.

### Example 7: Curd and Cheesemaking with Reconstituted Micelles/Liquid Colloid

At small scale (few mL), cheese was made in 24 well plates. Initial pH was recorded, and 6.65% citric acid solution was titrated in increments until the target pH of 5.1-5.2 was reached. A 0.15% rennet solution was added at 1.36% of the volume of the reconstituted liquid colloid and mixed gently. The samples were left undisturbed for approximately 30 minutes, or until a curd formed. The curds were then pipetted into microtubes and centrifuged for 2 minutes to separate the curd. The separated liquid was drained, and the curd was stretched by immersing in hot water (>60° C.). The cheese was stretched until smooth and homogenous, then shaped into a ball and weighed. For larger volumes, the process followed the same protocol with the exception of the centrifugation step. Instead, curds were drained using a mesh strainer lined with cheesecloth.

For full formulation cheese with fat, sugar and additional components, liquid colloid with induced micelles was warmed to 40° C. in a water bath. Fat was melted and blended with sugar until sugar was coated. If an emulsifier was used, it was also added to the fat/sugar blend. Then the warmed protein liquid colloid was poured into the fat/sugar mix and blended using a high shear mixer. Mixing time was dependent on the sample volume but ranged from 1-5 mins. The mixture was then passed through an Avestin Emulsiflex C-5 homogenizer at 5000 psi for 1 pass. Acidification and renneting are then performed as described above. FIGS. 7A and 7B show the curd and cheese formed from liquid colloid from Example 6 comprising micelle compositions A to F.

### Example 8: Curd and Cheese Making in Simulated Milk Ultrafiltrate (SMUF) “Milk Serum”

The above curd and cheese making protocol from Example 7 was performed with liquid colloid compositions with different salt conditions A to F, and an additional composition G (with no salts). The results are summarized in the Table 4 below.

TABLE 4
Curd and cheese making results

| Sample | Start pH | End pH | Acidification | Curd | Cheese |
|---|---|---|---|---|---|
| A | 6.35 | 5.01 | Some crash | ++ | stretchy |
| | | | out | | |
| B | 6.24 | 5.32 | Some crash | − | stretchy |
| | | | out | | |
| C | 6.31 | 5.24 | Some crash | − | stretchy |
| | | | out | (some crashed | |
| | | | | out bits) | |
| D | 6.34 | 5.31 | Some crash | − | stretchy |
| | | | out | | |
| E | 6.35 | 5.31 | Some crash | − | stretchy |
| | | | out | | |
| F | 6.12 | 5.29 | Some crash | ++ | stretchy |
| | | | out | | |
| G | 6.32 | 5.14 | Some crash | +/− | stretchy, |
| | | | out | | very small |

### Example 9: Casein Micelles/Liquid Colloid Reconstitution, Curd and Cheesemaking Using Alpha Casein and Kappa Casein

Alpha-casein and kappa casein in these experiments were purchased as a lyophilized powder from Sigma-Aldrich. The standard amounts of protein used in the micelle/liquid colloid forming experiments were 1.4% (0.5× milk concentration for casein), 2.8% (1× milk concentration for casein), or 3.2% (1× milk concentration for total protein, w/v) as also shown in Table 5. Unless otherwise noted, 15% of the total protein (by mass) was kappa casein, while 85% of the total protein was alpha casein.

TABLE 5
Protein concentrations

| | [alpha casein] | [kappa casein] |
|---|---|---|
| % Protein (total) (w/v) | (mg/ml) | (mg/ml) |
| 1.4 | 11.9 | 2.1 |
| 2.8 | 23.8 | 4.2 |
| 3.2 | 27.2 | 4.8 |

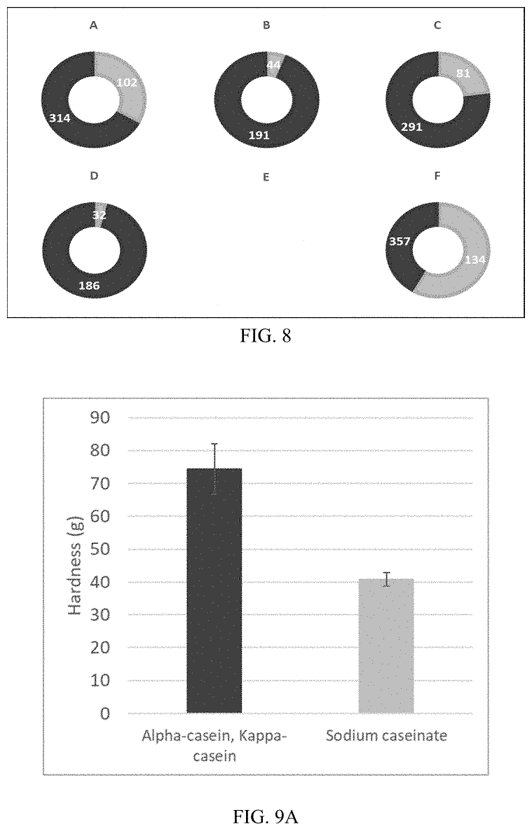

For 1.4% protein liquid colloid, the alpha casein and kappa casein were added sequentially to water and stirred until fully dissolved. In some experiments, the mixture was incubated at room temperature overnight. Calcium, phosphate, and citrate were then titrated to final concentrations from Table 3. The salt addition schedule and the subsequent particle size measurement method was performed similar to set forth in Example 6. Results of the particle sizing are shown in FIG. 8 and the labelling of the doughnut charts is the same as described in Example 6. Particle size data displayed only minor variations between each condition, and none showed any major aggregation.

Micelle/liquid colloid reconstitution and cheesemaking was also tested at 2.8% and 3.2% final protein concentrations as shown in Table 7, using the salt conditions in Table 6. Initial pH was about 6.0 and final pH was about 5.2. For cheese making citric acid and rennet were added as per Example 7.

TABLE 6
Final salt concentrations (mM) and protein concentrations (%)

| | 1.4% protein | 2.8% protein | 3.2% protein |
|---|---|---|---|
| Calcium (CaCl2) | 18.5 | 30.85 | 30.85 |
| Phosphate (K2PO4) | 12.4 | 16.5 | 16.5 |
| Citrate (K3 citrate) | 6.16 | 8.2 | 8.2 |

TABLE 7
Curd and cheese making results

| | | | | | Yield of | |
|---|---|---|---|---|---|---|
| | | | | | cheese | |
| | Volume | | | Cheese | per gram | |
| Composition | (ml) | Curd | Stretching | weight | protein | A575 |
| Alpha + | 2 | Whole | Easy to | 0.0742 | 1.70 | 1.94 |
| Kappa 2.8% | | curd | stretch | | | |
| Alpha + | 2 | Whole | Easy to | 0.0963 | 2.21 | 2.374 |
| Kappa 3.2% | | curd | stretch | | | |

### Scaled Up Alpha Casein+Kappa Casein Cheese for Tasting and Texture Analysis (in Full Formulation)

This experiment was done at a 30 mL scale using sodium caseinate (purchased from Sigma-Aldrich) as a control. Micelles were induced in both protein mixes (alpha and kappa casein, vs sodium caseinate), then used in the full formulation shown in Table 8 below.

TABLE 8
Cheese compositions

| Ingredient | Concentration (%) | Mass (g) |
|---|---|---|
| 2.3% protein | 95.4 | 30.00 |
| coconut oil | 1.0 | 0.31 |
| margarine | 1.0 | 0.31 |
| maltose | 3.0 | 0.79 |
| lecithin | 0.1 | 0.03 |
| water | 0 | 0 |
| total | 100% | 31.45 |

Curd and cheese making was performed with the above composition (Table 8) where the protein was alpha+kappa casein or sodium caseinate as a control. Starting pH was about 6.0 and final pH was about 5.1-5.2. Citric acid and rennet were added as per Example 7. Results are presented in Table 9.

TABLE 9
Curd and cheese making results

| | Sodium caseinate | α casein + κ casein |
|---|---|---|
| Curd properties | Disrupted curd, some protein | Whole curd |
| | crash out | |
| Stretching | good | good |
| Cheese weight | 1.17 | 1.76 |
| Cheese yield | 1.73 | 2.6 |

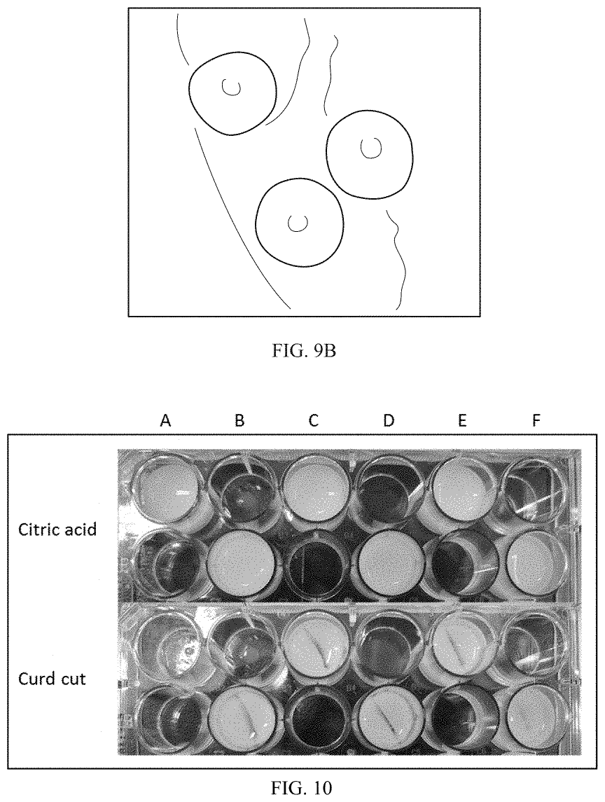

Alpha+kappa casein yielded more cheese and a better-quality curd, but with similar texture to the sodium caseinate cheese. The sodium caseinate cheese had a strong off-flavor, while the alpha+kappa cheese did not. Both cheeses were measured on the texture analyzer in triplicate and the results are shown in FIGS. 9A and 9B. While the cheese from alpha+kappa casein had a firmer texture, overall it was very similar to sodium caseinate cheese. This demonstrates that alpha+kappa caseins can form micelles/liquid colloid, dairy curd and dairy cheese and that beta casein is not necessary for these functions.

### Example 10: Casein Micelles/Liquid Colloid Reconstitution, Curd and Cheesemaking Using Alpha Casein (Dephosphorylated/Hypophosphorylated) and Kappa Casein

The proteins used for this experiment were dephosphorylated alpha casein and kappa casein (both from Sigma-Aldrich). The phosphorylation state of this alpha casein (marketed as dephosphorylated alpha casein) was assessed by Neutral-Urea-Triton PAGE, an established method for resolving the phosphospecies of individual proteins. The system uses Urea as a denaturant and is run at a neutral pH. This assessment demonstrated that the dephosphorylated alpha casein protein has an average of 1-2 phosphates remaining on a majority of the protein, and a small amount of protein with a greater level of phosphorylation. Hence, this protein is hypophosphorylated, meaning it has substantially reduced phosphorylation compared to milk alpha casein (1-2 phosphates form predominant vs 8-9 phosphates form predominant).

Hypophosphorylated alpha casein and kappa casein proteins were used in the amounts shown above in Table 5 and reconstituted into micelles/liquid colloid by adding them sequentially to water and stirred until fully dissolved. The mixture was then treated as described in Examples 7 and 8 and evaluated under similar salt conditions and protein concentrations.

Particle size was measured as set forth in Example 6. Curd and cheese making were performed using the methods as set forth in Examples 7 and 8. Hypophosphorylated alpha+kappa showed lesser monomer to micelle conversion efficiency than alpha+kappa, as seen by lowered turbidity (A400). Hypophosphorylated alpha+kappa produces somewhat looser micelles in general when compared to alpha and kappa or alpha, beta and kappa, but still within the range of native micelle sizes from milk (150-500 nm). Hypophosphorylated alpha+kappa did not exhibit any major aggregation.

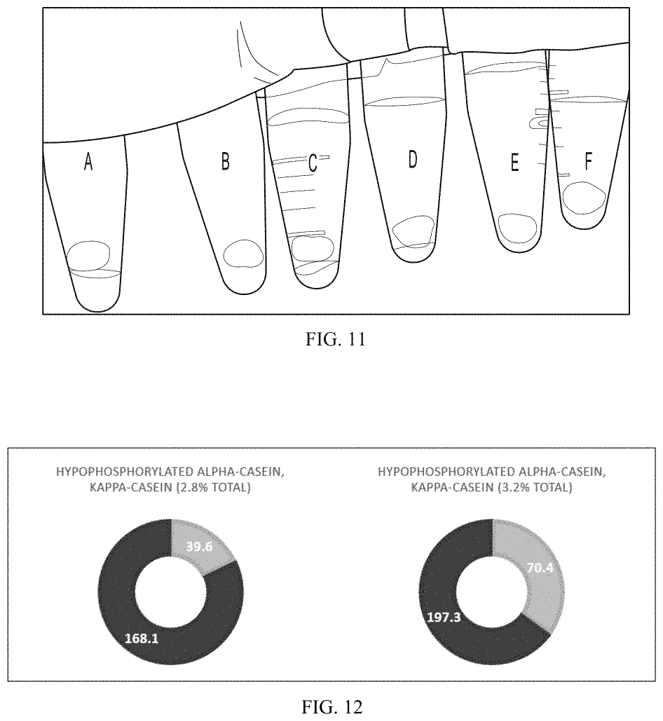

Cheesemaking results for hypophosphorylated alpha+kappa liquid colloid with acidification and with rennet are shown in FIG. 10, and a closer visual comparison of the cheese balls in FIG. 11.

Hypophosphorylated alpha and kappa micelles/liquid colloid were also evaluated at higher protein concentrations in salt conditions described in Example 9. Particle size for these conditions is shown in FIG. 12. The two concentrations of proteins (2.8% and 3.2%) gave whole curds with some liquid on top and the curds were easy to stretch. The cheese weight was 0.0467 grams, providing a yield of 1.07 g cheese/g protein for the 2.8% sample. The cheese weight was 0.0899 grams, providing a yield of 2.06 g cheese/g protein for the 3.2% sample. The A575 was 0.054 for the 2.8% sample and 0.038 for the 3.2% sample. Neither sample exhibited any aggregation.

### Example 11: Casein Micelles/Liquid Colloid Reconstitution, Curd and Cheesemaking Using Alpha Casein and Kappa Casein (Deglycosylated)

Deglycosylated kappa casein was generated from lyophilized kappa casein (Sigma-Aldrich). This was accomplished with trifluoromethanesulfonic acid (TFMS), which selectively deglycosylates proteins without significant protein degradation (Sojar and Bahl, 1987, A chemical method for the deglycosylation of proteins, Archives of Biochemistry and Biophysics; Electricwala et al, A Rapid and Improved Chemical Method for Deglycosylation of Glycoproteins, Sigma Aldrich).

The ProQ Emerald300 glycoprotein staining kit was used to detect the glycosylation of kappa casein, and to confirm that the deglycosylation was successful. The results indicated that the glycoprotein signal is eliminated (>95%) after deglycosylation reaction on the casein protein, meaning kappa casein was successfully deglycosylated.

Micelles/liquid colloid reconstitution experiments were performed using the 1.4% protein protocol (as described in Examples 6 and 9) as a starting point with 2× and 3× kappa casein concentrations tested while holding the alpha casein concentration constant. Deglycosylated kappa casein was stored in citric acid, and after mixing the kappa casein and alpha casein, the citric acid was neutralized by stoichiometric addition of NaOH. The added citrate amount was then reduced concomitantly from the total citrate required for the fixed additions schedule.

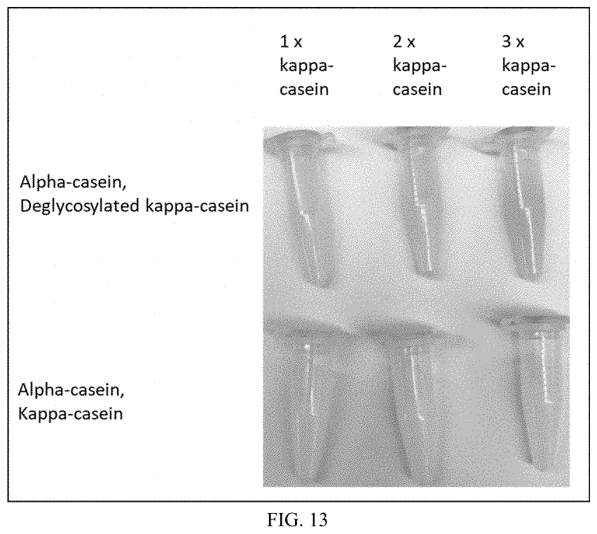

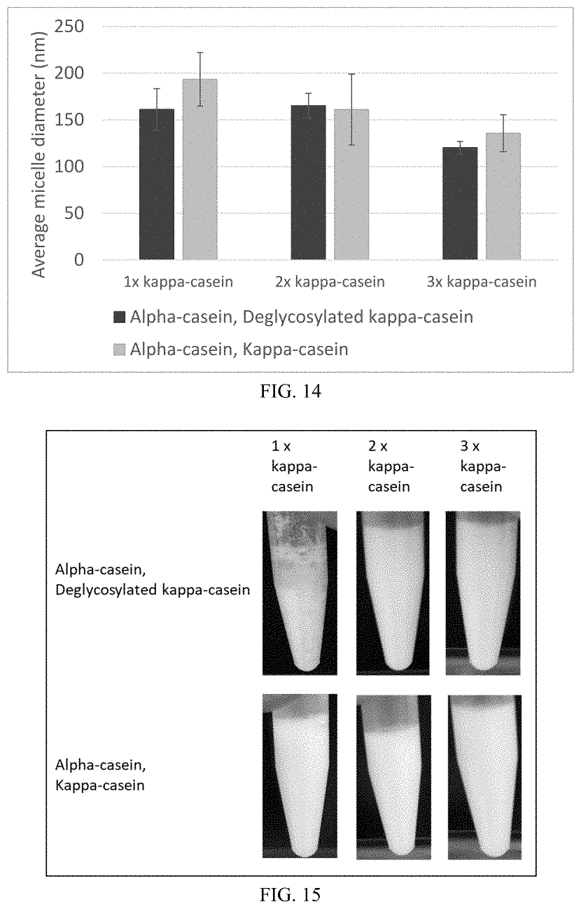

After micelles/liquid colloid were formed, each sample was examined visually, shown in FIG. 13, and the turbidities were assayed by absorbance at 525 nm as shown in Table 10 below. The average particle size of the micelle peaks, measured as described in Example 6, is shown in FIG. 14. The error bars indicate the standard deviation of the three replicate measurements.

TABLE 10
Turbidity results

| | 1X kappa casein | 2X kappa casein | 3X kappa casein |
|---|---|---|---|
| Alpha casein, | 1.4 | 3.2 | 1.7 |
| Deglycosylated | | | |
| kappa casein | | | |
| Alpha casein, | 4.1 | 1.9 | 1.1 |
| Kappa casein | | | |

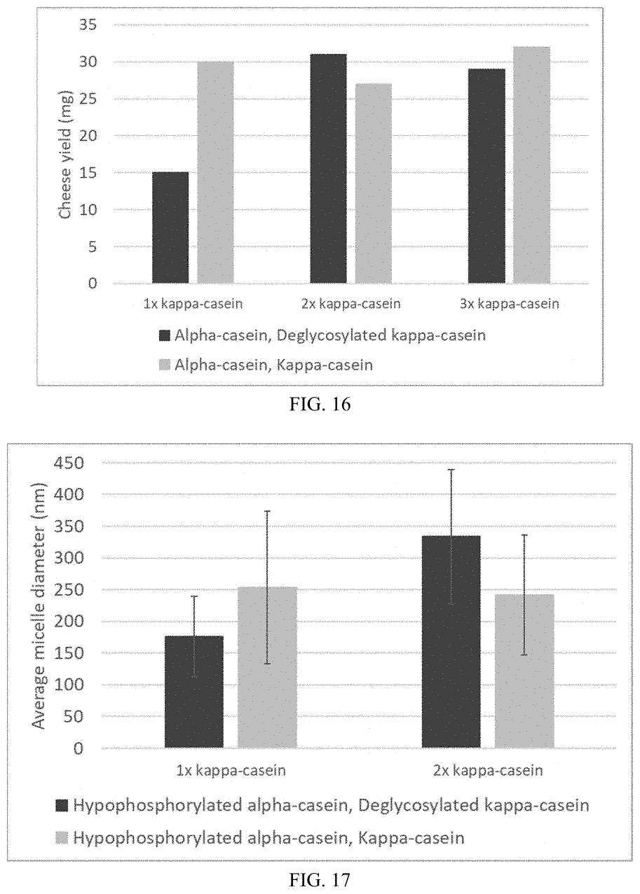

After acidification and renneting for cheese formation as described in Example 8, the different samples were assayed visually (FIG. 15) and for yield from 1 ml of liquid colloid (FIG. 16). All samples formed good curd, having a gel strong enough to be inverted without deforming except for the 1× deglycosylated kappa casein where the protein crashed out of solution. The curd in all of these conditions had good stretch and melted well.

### Example 12: Casein Micelles/Liquid Colloid Reconstitution, Curd and Cheesemaking Using Alpha Casein (Dephosphorylated/Hypophosphorylated) and Kappa Casein (Deglycosylated)

The methods for this example are the same as those used in Example 11, except that hypophosphorylated alpha casein was used in place of alpha casein, and only the 1× and 2× kappa casein conditions were tested. After micelle/liquid colloid formation, the turbidity (A525) was assayed and the results are shown in the table below.

TABLE 11
Turbidity Results

| | 1X kappa casein | 2x kappa casein |
|---|---|---|
| Hypophosphorylated alpha casein, | 2.3 | 11.23 |
| Deglycosylated kappa casein | | |
| Hypophosphorylated alpha casein, | 1.82 | 8.34 |
| Kappa casein | | |

The average particle size of the micelle peaks is shown in FIG. 17. The error bars indicate the standard deviation of the three replicate measurements. The curd yields from 1 ml of micelles formed in each condition were relatively similar and the curd in all conditions had good stretch and melted well.

### Example 13: Casein Micelles/Liquid Colloid Reconstitution, Curd and Cheesemaking Using Recombinant Alpha-S1-Casein (Dephosphorylated) and Kappa Casein

Micelles/liquid colloid were formed using recombinant alpha-S1-casein with kappa casein (Sigma-Aldrich). 1.4% protein was used, either with 1× or 2× kappa casein. The following salts were used: 27 mM calcium chloride, 22 mM disodium phosphate, 10 mM trisodium citrate. The reaction was started with a solution containing the alpha-S1-casein, kappa casein, trisodium citrate, and half of the disodium phosphate. Calcium chloride and the other half of disodium phosphate were added in nine additions in total, where the first addition comprised only a fraction of calcium and other additions equal fractions of calcium and phosphate.

The turbidities of the different conditions were as shown in Table 12.

TABLE 12
Turbidity results (A525)

| | 1x kappa casein | 2x kappa casein |
|---|---|---|
| Recombinant alpha-s1-casein, | 2.81 | 2.68 |
| kappa casein | | |

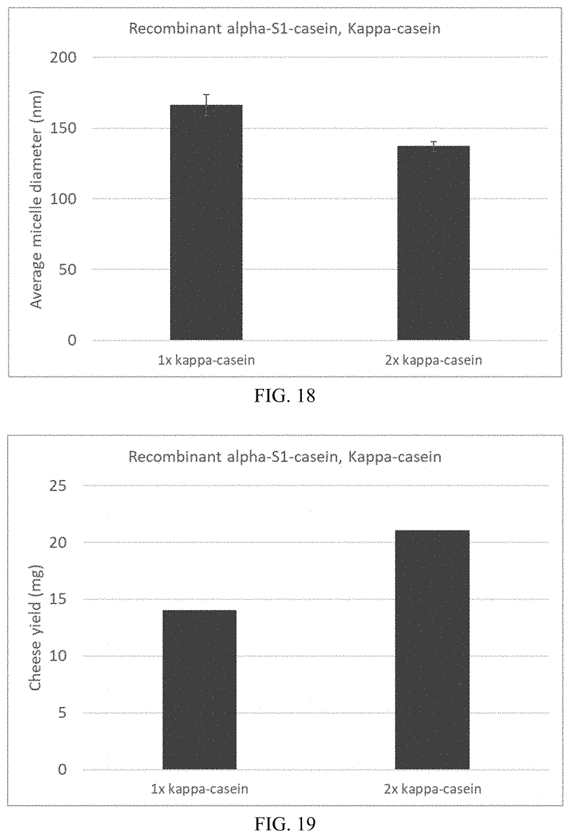

The average particle size of the micelle peaks is shown in FIG. 18. The error bars indicate the standard deviation of the three replicate measurements. Cheese yield for each condition is shown in FIG. 19.

The curd had the properties shown in Table 13 when being stretched.

TABLE 13
Curd stretch properties

| | 1x kappa casein | 2x kappa casein |
|---|---|---|
| Recombinant alpha-s1-casein, | Smooth stretch, | Smooth stretch, |
| kappa casein | slightly stiff | slightly stiff |

### Example 14: Casein Micelles/Liquid Colloid Reconstitution, Curd and Cheesemaking Using Recombinant Alpha-S1-Casein (Dephosphorylated) and Kappa Casein (Deglycosylated)

Micelles/liquid colloid were formed using recombinant alpha-S1-casein with deglycosylated kappa casein (as per Example 11). 1.4% protein was used, either with 1× or 2× kappa casein. The following salts were used: 27 mM calcium chloride, 22 mM disodium phosphate, and 10 mM trisodium citrate. The reaction was started with a solution containing the alpha-S1-casein, deglycosylated kappa casein, trisodium citrate, and half of the disodium phosphate. Micelles were formed as explained in Example 13. The turbidities of the different conditions are shown in Table 14.

TABLE 14
Turbidity results (A525)

| | 1x kappa casein | 2x kappa casein |
|---|---|---|
| Recombinant alpha-s1-casein, | 2.70 | 2.54 |
| deglycosylated kappa casein | | |

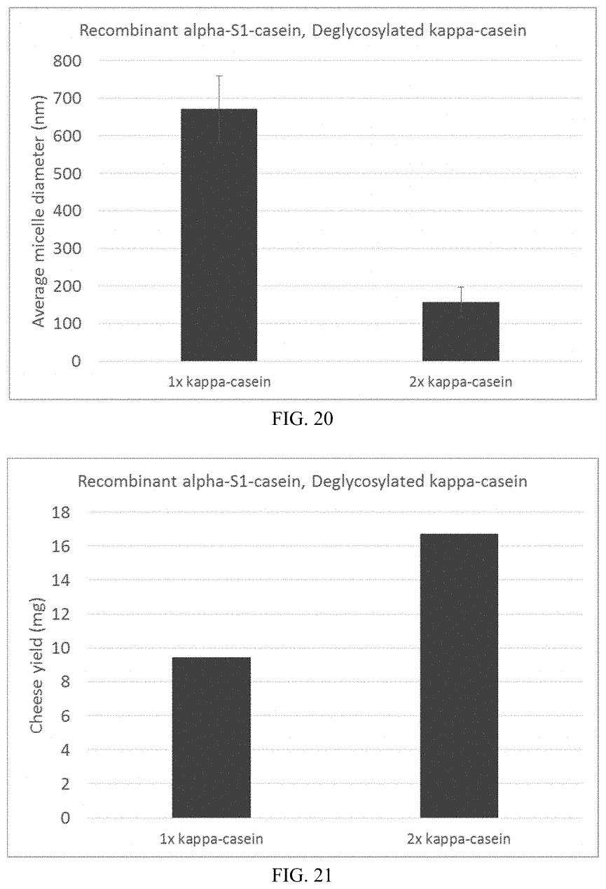

The average particle size of the micelle peaks is shown in FIG. 20. The error bars indicate the standard deviation of the three replicate measurements. Cheese yield for each condition is shown in FIG. 21. The curd had the properties indicated in Table 15 when being stretched.

TABLE 15
Curd stretch properties

| | 1x kappa casein | 2x kappa casein |
|---|---|---|
| Recombinant alpha-s1-casein, | Stretchy, sticky | Stretchy, sticky |
| deglycosylated kappa casein | | |

What is claimed is:

1. An edible composition comprising recombinant kappa casein protein,
wherein at least one property of the edible composition selected from the group consisting of texture, stretch, taste, melt, and hardness, is comparable to a dairy-derived edible composition, and
wherein 100% of casein protein in the edible composition is the recombinant kappa casein protein.

2. The edible composition of claim 1, wherein the edible composition comprises a coagulated colloid.

3. The edible composition of claim 2, wherein the coagulated colloid is derived from a liquid colloid.

4. The edible composition of claim 1, wherein the recombinant kappa casein protein lacks one or more post-translational modifications as compared to animal-derived kappa casein protein.

5. The edible composition of claim 4, wherein the one or more post-translational modifications is selected from the group consisting of: glycosylation, phosphorylation, and both.

6. The edible composition of claim 1, wherein the recombinant kappa casein protein is a recombinant form of a bovine, ovine, caprine, camel, equine, buffalo, or human kappa casein.

7. The edible composition of claim 1, wherein the kappa casein protein has an amino acid sequence comprising any one of SEQ ID NOs: 27-40, or a variant thereof having at least 95% sequence identity to any one of SEQ ID NOs: 27-40.

8. The edible composition of claim 1, wherein the edible composition lacks any animal-derived proteins.

9. The edible composition of claim 1, wherein the recombinant kappa casein protein is produced from a bacterial host cell.

10. A cheese composition comprising the edible composition of claim 1.

11. The cheese composition of claim 10, wherein the cheese composition comprises fat, sugar, or both.

12. The cheese composition of claim 11, wherein the cheese composition has a fat content of about 0% to about 50%, and the fat is free from animal-derived fats.

13. The cheese composition of claim 11, wherein the cheese composition has a sugar content of about 0% to about 10%, and the sugar is derived from a plant-based source.

14. The cheese composition of claim 10, wherein the cheese composition is capable of melting when heated.

15. The cheese composition of claim 10, wherein the cheese composition is capable of browning when heated.

16. An edible colloid, comprising a recombinant kappa casein protein, wherein the recombinant kappa casein protein is present in a colloidal form and is 90% to 100% of the protein content of the colloidal form.

17. The edible colloid of claim 16, wherein the recombinant kappa casein protein is 100% of the casein protein in the colloidal form.

18. The edible colloid of claim 16, wherein the colloidal form is a liquid colloid.

19. The edible colloid of claim 16, wherein the colloidal form is a coagulated colloid.

20. The edible colloid of claim 16, wherein the recombinant kappa casein protein lacks one or more post-translational modifications as compared to animal-derived kappa casein protein.

21. The edible colloid of claim 16, wherein the recombinant kappa casein protein has an amino acid sequence comprising any one of SEQ ID NOs: 27-40, or a variant thereof having at least 95% sequence identity to any one of SEQ ID NOs: 27-40.

22. The edible colloid of claim 16, wherein the edible colloid lacks any animal-derived proteins.

23. The edible colloid of claim 16, wherein the recombinant kappa casein protein lacks any casein protein other than the recombinant kappa casein protein in the colloidal form.

24. The edible colloid of claim 16, wherein the recombinant kappa casein protein is a recombinant form of a bovine, ovine, caprine, camel, equine, buffalo, or human kappa casein protein.

25. A cheese composition comprising the edible colloid of claim 16.

26. A yogurt composition comprising the edible colloid of claim 16.

27. A dairy-like composition comprising the edible colloid of claim 16.
